(19) United States
(12) Patent Application Publication
(10) Pub. No.: US 2012/0108452 A1
(43) Pub. Date: May 3, 2012

(54) Semi-Invasive Method for Characterizing Lung Injury

(75) Inventors: David Neujahr, Atlanta, GA (US); Aminu Mohammed, Atlanta, GA (US); Jeffrey Rossio, Frederick, MD (US)

(73) Assignee: LIFE TECHNOLOGIES CORPORATION, Carlsbad, CA (US)

(21) Appl. No.: 13/044,496

(22) Filed: Mar. 9, 2011

Related U.S. Application Data
(60) Provisional application No. 61/312,186, filed on Mar. 9, 2010.
(60) Provisional application No. 61/433,446, filed on Jan. 17, 2011.

(51) Int. Cl. C40B 30/04; G01N 33/566

(52) U.S. Cl. 506/9; 435/7.92

(57) ABSTRACT

Described and disclosed are methods for determining, predicting, diagnosing, treating, and monitoring lung diseases, including bronchiolitis obliterans syndrome and acute cellular rejection in a lung transplant recipient by measuring chemokine levels in bronchoalveolar lavage (BAL) samples. The chemokine CXCL10 is measured in combination with at least one analyte selected from the group consisting of IL1RA, CXCL11, MCP-1, CXCL9, RANTES, IL-13, IL-17, IL-22, fractalkine, and eotaxin; and/or biomarkers. The present teachings also relate to methods, treatment decisions and kits for detecting and monitoring onset of lung transplant rejection in advance of clinically recognized symptoms.

### FIELD

In general, the present teachings relate to methods of diagnosing, predicting and monitoring lung diseases, including bronchiolitis obliterans syndrome and acute cellular rejection. In particular, the present teachings relate to detection, prediction and monitoring of lung transplant rejection diseases by detection of CXCL9 and CXCL10 in combination with additional chemokines and/or biomarkers in bronchioalveolar lavage samples. The present teachings also relate to methods and kits for detecting and monitoring onset of lung transplant rejection in advance of onset of clinically-approved test parameters.

### BACKGROUND

Lung transplantation is a viable therapy for select patients with end-stage lung disease. In spite of surgical advances over the past two decades, outcomes in lung transplantation have lagged behind those of other solid organs. In particular, the 5-year survival rate following lung transplantation is less than 50% (Christie JD, et al. “The Registry of the International Society for Heart and Lung Transplantation: Twenty-sixth Official Adult Lung and Heart-Lung Transplantation Report-2009,” J Heart Lung Transplant 2009; 28:1031-49). The largest hurdle to long term survival and to continued improved quality of life following lung transplantation is bronchiolitis obliterans (BO), a form of chronic allograft rejection characterized by obliteration of the terminal bronchiole by fibrosis. Many putative factors have been shown to increase the risk of developing BO including ischemia reperfusion injury, infections, episodes of acute rejection, degree of antigenic mismatch between donor and recipient, and aspiration of gastric contents (reviewed in Weigt SS, et al. “Chronic allograft rejection: epidemiology, diagnosis, pathogenesis, and treatment,” Semin Respir Crit Care Med; 31:189-207). It is the known association with acute cellular rejection (ACR) early after transplant and the subsequent development of BO that has formed the rationale for periodic surveillance lung biopsy protocols which are performed by many centers around the world. These biopsies, performed during fiberoptic bronchoscopy procedures provide the clinician with samples that can be scored for rejection so that patients experiencing early rejection can be aggressively treated and subsequent BO averted. ACR can be clinically silent, and is a common phenomenon in the first post-transplant year. Post-transplant Surveillance can also lead to aggressively treating ACR early in the hope of decreasing the risk of OB in subsequent years.

Implicit in protocols that employ routine surveillance biopsy procedures are two important premises: One, that the small tissue samples obtained at biopsy give an accurate representation of the degree of immunologic injury happening within the lung, and two, that the knowledge of such processes is meaningful clinically with respect to future lung function. The practical use of surveillance lung biopsy is hampered by a small but finite risk related to performing multiple lung biopsies over time, a requirement for significant pathologic expertise in interpreting lung biopsies and an unquantifiable risk of false negative biopsy specimens (Glanville Ark. “Bronchoscopic monitoring after lung transplantation,” Semin Respir Crit Care Med; 31:208-21).

Surveillance bronchoscopy involves placing a bronchoscope into the lung(s) of a transplant recipient, performing a bronchioalveolar lavage (BAL) of the transplanted lung with instilled saline, and performing multiple forceps biopsies of the transplanted allograft. The lavage specimen is used to rule out infection through microbiologic techniques. The lung biopsy pieces are reviewed by a pathologist and scored for acute rejection. Lung biopsy is viewed as the gold standard for the detection of rejection. However, there are many limitations to its use. First, transbronchial lung biopsy is associated with medical risks such as hemorrhage and pneumothorax. The per procedure risk of clinically significant bleeding requiring intervention is about 5%, while the risk for pneumothorax requiring intervention is about 2%. Hence, lung transplant recipients who undergo multiple procedures in the post-transplant period are at high cumulative risk for an adverse outcome. Second, transbronchial biopsy is not feasible in some transplant recipients because of a high risk for bleeding, possibly due to the use of blood thinners or because of mild renal failure making the procedure contraindicated in as many as 30% of lung transplant recipients. Third, there is a delay between assessment of lung biopsy and diagnosis of rejection due to the considerable processing and staining of tissue required prior to assessment. Fourth, inter-rater variability among pathologists viewing the stained tissue confounds the reliability of lung biopsy with the potential to delay therapeutic intervention for ACR.

The present teaching are based on the hypothesis that better immune monitoring tools could enhance the ability of lung biopsy to project future risk of BO for patients. An untapped resource from a biomarker standpoint is the bronchoalveolar lavage fluid (BALF). This fluid, obtained by injecting saline into an entire region of the lung, and then aspirating that saline contains both a cellular component with multiple potential prognostic biomarkers as well as a supernatant which contains constituents which may be involved in recruitment of injurious cells. Among these constituents are the interferon gamma (IFNG) dependent chemokines CXCL9 and CXCL10 which have already been shown to be elevated in patients with existing BO as well as experimental lung rejection in animals (Agostini C., et al. “CXCR3 and its ligand CXCL10 are expressed by inflammatory cells infiltrating lung allografts and mediate chemotaxis of T cells at sites of rejection,” Am J Pathol 2001; 158:1703-11; Belperio J A, et al. “Critical role for CXCR3 chemokine biology in the pathogenesis of bronchiolitis obliterans syndrome,” J Immunol 2002; 169:1037-49; Belperio J A, et al. “Role of CXCL9/CXCR3 chemokine biology during pathogenesis of acute lung allograft rejection,” J Immunol 2003; 171:4844-52).

Additional work has shown that IL-13 is elevated in patients with chronic rejection, and that blocking the effects of IL-13 can diminish experimental fibrosis in mice (Keane M P, et al. “IL-13 is pivotal in the fibro-obliterative process of bronchiolitis obliterans syndrome,” J Immunol 2007; 178:511-9). Chemoattractants capable of increasing lung migration of monocyte/macrophage subsets into the lung have also been shown to be elevated in human lung transplant recipients at risk for the development of chronic rejection as well (Belperio J A, et al. “Critical role for the chemokine MCP-1/CCR2 in the pathogenesis of bronchiolitis obliterans syndrome,” J Clin Invest 2001; 108:547-56).

Therefore, methods which are less invasive, but which convey clinically relevant information in lung transplantation are needed. One tool to assess lung transplant rejection is the assessment of inflammatory chemokines within the BAL fluid in addition to, or instead of the use of lung biopsy. The analysis of BAL fluid offers several advantages over lung biopsy. First BAL can be obtained from virtually all lung transplant recipients without any significant risk for major adverse effects. Second, the assessment of BAL fluid can be completed within hours of collection (in contrast to days with biopsy). Third, because BAL samples an entire region of lung (as opposed to random blind biopsies), BAL analysis can remove the possibility of sampling error and the potential to preclude false negative results due the subjectivity grading of rejection associated with lung biopsy. Fourth, because BAL analysis can result in detection of cells and chemokines with cut-off values, unlike lung biopsy, BAL analysis holds the promise of standardizing immune monitoring protocols across lung transplant programs. Therefore, there skill exists in the art the need for more specific, sensitive and consistent assessment of acute and chronic rejection in lung transplant recipients.

### SUMMARY OF SOME EMBODIMENTS TEACHINGS

In some embodiments, disclosed are methods for determining the status of a transplanted organ, such as lung, including chronic and/or acute cellular rejection in advance of FEV-1 or pathological indicators of lung rejection. In some embodiments the determining can include diagnosis, detecting, predicting future vitality, distinguishing and determining risk of lung rejection, rather it be chronic or acute cellular rejection verses nonrejection.

In some embodiments, the disclosed methods include contacting a body fluid sample, such as a BAL sample from a lung transplant recipient or a blood sample from a heart transplant recipient or a liver transplant recipient or a urine sample from a kidney transplant recipient with a reagent for the detection of at least one analyte selected from the group consisting of IL1RA, CXCL10, MCP-1, CXCL9, RANTES, IL-13, IL-17, IL-22, fractalkine, and eotaxin, IL-22, fractalkine, eotaxin; and detecting the presence of the analyte. In some embodiments the detection can be the detecting the presence or absence of at least one of the analytes or at least 15 pg/ml of at least one analyte.

In other embodiments, disclosed are methods for determining chronic and acute cellular rejection including contacting a BAL sample with a first reagent for detection of at least one chemokine receptor selected from the group consisting of CXCn, CCRn, CX₃CR1, and XC, wherein n is an integer from 1 to 10, detecting presence or absence of said at least one receptor from the group consisting of CXCn, CCRn, CX₃CR1, and XC in said biopsy sample using said reagent; and determining chronic or acute cellular rejection in said subject based on the result of said detecting.

In other embodiments, disclosed are kits for determination of transplant rejection having reagents for detection of CXCL10 and at least one analyte selected from the group consisting of IL1RA, MCP-1, CXCL9, RANTES, IL-13, IL-17, IL-22, fractalkine, and eotaxin. The kits may contain optional components such as wash solutions, buffers, beads, fluorescent tags, instructions, 96-well plates and a protocol.

### BRIEF DESCRIPTION OF THE DRAWINGS

FIGS. 1A-1D are graphs of BAL levels of CXCL10, IL1RA, CXCL9, and RANTES in lung rejection and nonrejection samples.

FIG. 2 is a graph of BAL CXCL10 levels during lung rejection and after administration of anti-rejection therapeutics.

FIG. 3A-3O are graphs comparing five scoring montages to detect acute rejection.

FIG. 4 is a graph of CXCL10 levels over time post-transplant in representative stable allograft recipients.

FIG. 5 is a graph of CXCL10 levels over time post-transplant in representative recipients with at least one ACR episode in the first post-transplant year.

FIG. 6 is a graph of CXCL9 levels over time post-transplant in representative stable allograft recipients.

FIG. 7 is a graph of CXCL9 levels over time post-transplant in representative recipients with at least one ACR episode in the first post-transplant year.

FIG. 8 is a graph of IR-1RA levels over time post-transplant in representative stable allograft recipients.

FIG. 9 is a graph of IR-1RA levels over time post-transplant in representative recipients with at least one ACR episode in the first post-transplant year.

FIG. 10 is a graph of RANTES levels over time post-transplant in representative stable allograft recipients.

FIG. 11 is a graph of RANTES levels over time post-transplant in representative recipients with at least one ACR episode in the first post-transplant year.

FIG. 12 is a graph of IL-17 levels over time post-transplant in representative stable allograft recipients.

FIG. 13 is a graph of IL-17 levels over time post-transplant in representative recipients with at least one ACR episode in the first post-transplant year.

FIG. 14 is a graph of MCP 1 levels over time post-transplant in representative stable allograft recipients.

FIG. 15 is a graph of MCP 1 levels over time post-transplant in representative recipients with at least one ACR episode in the first post-transplant year.

FIG. 16A-B are graphs of CXCL10 and CXCL9 levels, respectively, in a recipient who developed bronchiolitis obliterans in the first post-transplant year.

FIG. 17A-B are graphs of IR-1RA and RANTES levels, respectively, in a recipient who developed bronchiolitis obliterans in the first post-transplant year.

FIG. 18A-B are graphs of IL-17 and MCP 1 levels, respectively, in a recipient who developed bronchiolitis obliterans in the first post-transplant year.

FIG. 19 A-G are tables comparing BAL sample CXCL10 levels and microbiology findings with % FEV-1 and Biopsy scoring in recipients in the first post-transplant year.

FIG. 20 is a graph of the relationship of BAL CXCL10 and MFI of BAL CD8+ Granzyme B mean.

FIG. 21 is graph of the relationship between BAL CXCL10 and BAL CXCR3+CD8+ cells.

FIG. 22 is a graph of survival of lung transplant patients with high or low levels of BAL CXCL9 or CXCL10, event free survival indicating freedom from death or listing for retransplantation.

FIG. 23 is a graph of survival of lung transplant patients with high or low levels of BAL CXCL9 or CXCL10, event free survival indicating freedom from diagnosis of BOS or histologic OB.

FIG. 24 illustrates AUC for CXCL10 that negatively correlates with improvement in lung function after transplant.

FIG. 25 illustrates CXCL9 and CXCL10 BALF levels that are elevated in the setting of ACR compared to quiescent biopsies.

FIG. 26 illustrates anatomic compartment of staining for gamma dependent chemokines.

### DESCRIPTION OF EXEMPLARY EMBODIMENTS

For the purposes of interpreting of this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. For illustration purposes, but not as a limitation, “X and/or Y” can mean “X” or “Y” or “X and Y”. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”. The term “and/or” means one or all of the listed elements or a combination of any two or more of the listed element.

The section headings used herein are for organizational purposes only and are not to be construed as limiting the described subject matter in any way. All literature cited in this specification, including but not limited to, patents, patent applications, articles, books, and treatises are expressly incorporated by reference in their entirety for any purpose. In the event that any of the incorporated literature contradicts any term defined herein, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The practice of the present teachings may employ conventional techniques and descriptions of organic chemistry, polymer technology, molecular biology (including recombinant techniques), cell biology, biochemistry, and immunology, which are within the skill of the art. Such conventional techniques include oligonucleotide synthesis, hybridization, extension reaction, and detection of hybridization using a label. Specific illustrations of suitable techniques can be had by reference to the example herein below. However, other equivalent conventional procedures can, of course, also be used. Such conventional techniques and descriptions can be found in standard laboratory manuals and texts such as Molecular Cloning: A Laboratory Manual (all from Cold Spring Harbor Laboratory Press, 1989), Current Protocols in Immunology” Series Editor Richard Coico, Most recent update November 2009. John Wiley and Sons, Inc., Malden, Mass., Janeway's Immunobiology, Seventh Edition” Editors Kenneth Murphy, Paul Travers, and Mark Walport. 2007. Garland Science, New York, N.Y., Lehninger, Principles of Biochemistry 3^{rd} Ed., W. H. Freeman Pub., New York, N.Y., and Berg et al. (2002) Biochemistry, 5^{th} Ed., W. H. Freeman Pub., New York, N.Y., all of which are herein incorporated in their entirety by reference for all purposes.

The term “allele” as used herein refers to a genetic variation associated with a gene or a segment of DNA, i.e., one of two or more alternate forms of a DNA sequence occupying the same locus.

The term “transplant” as used herein refers to the implantation of an organ from one organism into another organism with the expectation that the recipient of the implanted organ will utilize the organ for its intended physiological functions. Examples of transplanted organs include, but are not limited to, kidney, lung, liver and heart.

The term “rejection” as used herein refers to a pathological activity resulting in the degradation, functional failing or necrosis of a transplanted organ. An example of a rejection process is an immunological response to a transplanted tissue or organ such as graph verses host and host verse graph.

The term “nonjection” as used herein refers to absence of cellular rejection based on the International ISHLT scoring system (see Revision of the 1996 working formulation for the standardization of nomenclature in the diagnosis of lung rejection. S. Stewart, S A Yousem, et al. J Heart Lung Transplant 2007; 12:1229-42).

The term “chronic rejection” as used herein refers to bronchiolitis obliterans syndrome in lung transplant recipients.

As used herein, “Bronchiolitis Obliterans” refers to obliteration of the terminal airway with fibrotic material. Reference: “A working formulation for the standardization of nomenclature and for clinical staging of chronic dysfunction in lung allografts, International Society for Heart and Lung Transplantation” JD Cooper et al. J Heart Lung Transplant 1993; 12:713-6.

The term “acute rejection” as used herein refers to acute cellular rejection in a transplant recipient's transplanted tissue or organ.

The term “bronchoalveolar lavage (BAL)” as used herein refers to the process of introducing a solution into the bronchoalveoli of the lung and then recovering the solution from the lung.

The term “contacting” as used herein refers to the mixing together or combining of a BAL eluate or extract with a reagent including, but not limited to, an antibody, antigen, dye, or protein.

The term “detecting” as used herein refers to the observing, registering on an instrument or receiving a signal from the mixture of a reagent and a sample to be tested when analyzing the sample for the presence or absence of an analyte or biomarker. Detection can involve a comparison of an analyte's or biomarker's concentration to the concentration of the analyte or biomarker in a baseline reference sample known to lack the analyte or biomarker being evaluated.

The term “determining” as used herein refers to analyzing for the presence or absence of an analyte or biomarker by making a measurement, observation or calculation of an amount of an endpoint or reaction between a reagent and a sample.

The term “diagnosing” as used herein refers to making a clinical determination of a physiological and/or pathological process in a subject from whom a sample has been tested.

The term “subject” as used herein refers to any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject. As used herein, in some embodiments “chemokines” are CXCR3 chemokines which bind to the CXCR3 receptor, including, but not limited to, CXCL9, CXCL10, and CXCL11. In other embodiments, chemokines” are CCL class chemokines, which bind to the CCR-5 receptor. Exemplary CCL class chemokines include, but are not limited to, MIP-Ia, MIP-3a, and MIP-Ib.

The term “sample” is used in its broadest sense. In one sense, it is meant to include a specimen or culture obtained from any source, as well as biological and environmental samples. Biological samples may be obtained from animals (including humans) and encompass body fluids, fluids, solids, tissues, and gases. Biological samples include tears, saliva, sputum, urine and blood products, such as plasma, serum and the like. Such examples are not however to be construed as limiting the sample types applicable to the present teachings. Lavage samples are also included as a sample, including BAL samples.

The term “frequency” as used herein refers to the percentage of a cell or signal detected relative to a parent population from which the measured frequency represents a subset of the parent. For example, for the measurement of GranzymeB hi frequency, the measurement obtained represents the percentage of CD8 lymphocytes which express a detectable level of the marker GranzymeB divided by the entire population of CD8 lymphocytes.

The term “mean fluorescent intensity (MFI)” as used herein refers to the arithmetic average of the channel fluorescence of each of N events divided by N.

The term “presence” as used herein refers to detection of an analyte, marker or biomarker in a sample undergoing evaluation, testing, monitoring or analysis. The detection for the presence of the analyte, marker or biomarker is seen as present when the concentration of the analyte, marker or biomarker is above that of a reference sample in which the analyte, marker or biomarker is known to not be present.

The term “absence” as used herein refers to the lack of detection of an analyte, marker or biomarker in a sample undergoing evaluation, testing, monitoring or analysis. The lack of detection of an analyte, marker or biomarker can be due to conditions in which the concentration of the analyte, marker or biomarker cannot be determined to be greater than that of a reference negative sample.

The term “risk” as used herein refers to the predisposition to present clinically with an episode of Acute Cellular Rejection (ACR).

The term “risk assessment” as used herein refers to the evaluation of clinical or laboratory findings that provide indicators for the development of ACR.

The term “increased risk” as used herein refers to the increased likelihood of a lung transplant recipient to develop ACR post-transplant.

The term “risk of acute lung transplant rejection” as used herein refers to the pretest probability that any lung transplant recipient undergoing a bronchoscopic procedure in which lung tissue is recovered for histologic assessment will have a diagnosis of acute cellular rejection as defined by the ISHLT scoring criteria.

The term “vitality” as used herein refers to the transplanted lung's function as referenced to a Nomogram adapted from a set of normal individuals. An example is the Crapo Nomogram. The recipient's spirometric measures, including but not limited to, forced expiratory flow in 1-second (FEV-1) would be measured. “Future vitality” refers to the transplanted lung's function as a measure of the recipient's continued FEV-1 being at a level where there is not a greater than 20% reduction in FEV-1 compared to peak FEV-1.

The term “determining a treatment course of action” as used herein refers to using the results of a test to make a decision on whether to maintain the current dosage and combination of immunosuppressive medications or to make alterations in the regimen based on the results of the test method.

The term “diagnosing acute lung transplant rejection” as used herein refers to the act of performing a bronchoscopy on a subject wherein transbronchial lung biopsy specimens are obtained and evaluated by histology according to the ISHLT scoring system.

The term “analyte” as used herein refers to a protein constituent from a biologic specimen. Examples of an analyte include, but are not limited to, a chemokine, an antigen, a cytokine, a chemokine receptor, and so on.

As used herein, in some embodiments “chemokines” include, but are not limited to, CXCL chemokines which bind to the CXCR3 receptor, including, but not limited to CXCL9, CXCL10, and CXCL11. In other embodiments, “chemokines” are CCL class chemokines, which bind to the CCR-5 receptor. Exemplary CCL class chemokines include, but are not limited to, MIP-Ia, MIP-3a, and MIP-Ib and others listed in Table 1.

The term “marker” as used herein refers to a cellular component, e.g., a granule, e.g., GranzymeB, to cell surface molecules, e.g., CD38, which not only designate cell surface proteins, but also allow for subsets of cells within parent populations such as CD38 positive cells among all CD8 positive cells, a macromolecule and so on.

The term “amount” as used herein refers to a measurement of the level, quantity, intensity, or number of the item being measured.

The term “longitudinal analysis” as used herein refers to repeated measurements made at predetermined time points post-transplant on a plurality of BAL samples from a subject.

The term “monitoring” as used herein refers to the systematic, periodic, random or intervals of performing a test, making an observation, and/or collecting a sample, and can be based on a predetermined clinical protocol applied to all subjects who undergo transplantation.

The term “measuring” as used herein refers to the action of quantitating the level, intensity or number of an item such that the item is expressed as an amount in terms of concentration (e.g., nanograms/milliliter), frequency (e.g., events of a subset/events of parent population), or arbitrary units such as MFI, where the measured attribute has a range of potential values.

The term “concurrent” as used herein refers to a simultaneous or adjacent event, issue, item or determination made in a sample when measuring, monitoring or observing at least two items for which an amount can be given to each item.

The term “infection” as used herein refers to the presence of an entity foreign to the host. that may trigger the host to elicit an immune response in response to the foreign entity. A bacterium, virus, protozoan, or fungus may be the infective agent of an infection. The origin of the infection may arise from outside of the host, from the transplanted allograft, or from reactivation of latent infection from within the host in the setting of immunosuppressant drugs (e.g., Cytomegalovirus reactivation).

The term “concurrent infection” as used herein refers to an infection process occurring in parallel or simultaneously with another pathological event, including but no limited to a transplant rejection.

The term “administration” as used herein refers to the giving of a treatment, a pharmaceutical, or a therapeutic to a subject.

The term “affixed” as used herein refers to the joining or securing, temporarily, semi-permanently, or permanently one item or items to another item or items.

The term “anti-rejection therapy” as used herein refers to the treatment(s), whether it be pharmaceutical, physical or therapeutic, alone or in any combination thereof, to a subject to preclude, avert, prevent, reverse or in response to a rejection event, a pathological determination, an immunological determination and the like. As used to refer to drug therapy, anti-rejection therapy refers to drugs from various pharmacologic classes: steroids, calcinurin-inhibitors, mTOR inhibitors, antimetabolites, and biologics. Anti-rejection therapy also refers to therapeutic efforts to alter the immune systems such as the uses of plasmapheresis, photopheresis, gastric fundoplication, lymphoid irradiation and total lymphoid irradiation.

The term “solid support” as used herein refers to a semi-flexible or inflexible item or surface upon which another item can be positioned upon, attached, affixed.

The term “immunoassay” as used herein refers to is a technique which measures a relevant analyte or biomolecule by binding an antibody to said analyte.

The term “reagent” as used herein refers to a biochemical, biological, or chemical solution, semi-solid, solid, powder, dispersion, slurry, or vapor.

The term “reagents for detection of at least one analyte” refers to reagents for the detection of a given analyte including, but not limited to, IL1RA, CXCL10 (IP10), MCP-1, CXCL9 (MIG), RANTES, IL-13, IL-17, IL-22, fractalkine, and eotaxin, IL-22, fractalkine, eotaxin, for example, in a BAL sample collected from a subject. In some embodiments, the reagent is an antibody for the CXCR3 ligand or other chemokine receptor ligands as listed in Table 1. In some embodiments, the reagents further comprise additional reagents for performing detection assays, including, but not limited to, controls, buffers, etc.

As used herein, the term “reagents for detection of a CCL chemokine” refers to reagents for the detection of a given CCL chemokine. Table 1 lists examples of CCL chemokines. For example, the reagent(s) can be used in the detection of a CCL chemokine in a BAL sample collected from a subject. In some embodiments, the reagent is an antibody specific for the CCL chemokine. In some embodiments, the reagents further comprise additional reagents for performing detection assays, including, but not limited to, controls, buffers, etc.

The term “ELISA” as used herein refers to an enzyme linked immunosorbant assay, a type of immunoassay, in which an analyte is quantitated by measuring a biochemical response detected on the complimentary antibody bound to the analyte.

The term “quantitative ELISA assay” as used herein refers to a subset of the ELISA assay in which a quantity of analyte, including but not limited to, concentration, is determined from the assay.

The term “flow cytometry” as used herein refers to a method and a process whereby cells within a sample can be detected and identified when transversing past a detector within an apparatus containing a detecting source and a flowing apparatus.

The term “fluorescently activated cell sorting assay” (FACS) as used herein refers to any assay suitable for use in cell sorting techniques (e.g., flow cytometry) that employs detection of fluorescent signals.

The term “quantitiative fluorescently activated cell sorting assay (qFAS)” as used herein refers to any assay suitable for use in cell sorting techniques (e.g., flow cytometry) that employs detection and quantification of fluorescent signals.

The present teachings provide methods for the prediction, determination, detection and diagnosis of lung diseases, including bronchiolitis obliterans and acute cellular rejection. In particular, the present teachings provide methods of determining and diagnosing lung disease based on the presence of chemokines, biomolecules and leukocyte subsets in fluid extracted from lungs, including but not limited to, BAL fluid. The present teachings further provide kits for detecting and monitoring lung diseases.

For example, the present teachings provide a novel, non-invasive method for detecting and correlating the presence of chemokines in BAL with chronic rejection and acute cellular rejection. The methods are a significant improvement in terms of decreased cost, rapid time to result, sensitivity and specificity, removal of the inherent subjectivity when accessing pathological samples, and minimal physical trauma to a recipient. The methods of the present teachings provide the further advantage of proactively changing, augmenting or simplifying anti-rejection chemotherapies in advance of clinical testing evidence of such need as is recommended by currently followed ISHLT protocols.

The current teachings result from a study to utilize multiplex ELISA of human lung transplant BALF assessing multiple candidate chemokines with respect to their ability to predict graft failure, chronic rejection, acute rejection, and peak lung function post-transplant. The study utilized an institutional review board approved tissue acquisition protocol actuating a commitment to store for later analysis any lung fluid obtained during clinical protocol driven bronchoscopy. The study assessed lung fluid obtained during clinical protocol driven bronchoscopy outcomes in 40 lung transplant patients in whom an entire year of BALF fluid was available for analysis. 7 candidate chemokines were chosen (CXCL9, CXCL10, MCP-1, IL1-RA, RANTES, IL-13, IL-17) and their concentrations measured in the first year post-transplant. These chemokines were chosen for analysis based on their high degree of detectability in previous pilot experiments (RANTES, IL1-RA, MCP-1) as well as a strong animal and human literature supporting their potential role (CXCL9, CXCL10, IL-13, IL-17). Overall, it was found that CXCL9 and CXCl10 exposure had the highest association with graft function and survival.

Many individual chemokines are known by a variety of names. Table 1 lists the known chemokines, other names known to one of skill in the art and their receptors.

TABLE 1

| | | Chemokine |
|---|---|---|
| Chemokines | Other name(s) for Chemokines | Receptor |
| CCL1 | I-309, TCA-3 | CCR8 |
| CCL2 | MCP-1 | CCR2, CCR2 |
| CCL3 | MIP-1a | CCR1 |
| CCL4 | MIP-1β | CCR1, CCR5 |
| CCL5 | RANTES | CCR5 |
| CCL6 | C10, MRP-2 | CCR1 |
| CCL7 | MARC, MCP-3 | CCR2 |
| CCL8 | MCP-2 | CCR1, CCR2B, |
| | | CCR5 |
| CCL9/CCL10 | MRP-2, CCF18, MIP-1? | CCR1 |
| CCL11 | Eotaxin | CCR2, CCR3, |
| | | CCR5 |
| CCL12 | MCP-5 | |
| CCL13 | MCP-4, NCC-1, Ckβ10 | CCR2, CCR3, |
| | | CCR5 |
| CCL14 | HCC-1, MCIF, Ckβ1, NCC-2, CCL | CCR1 |
| CCL15 | Leukotactin-1, MIP-5, HCC-2, | CCR1, CCR3 |
| | NCC-3 | |
| CCL16 | LEC, NCC-4, LMC, Ckβ12 | CCR1, CCR2, |
| | | CCR5, CCR8 |
| CCL17 | TARC, dendrokine, ABCD-2 | CCR4 |
| CCL18 | PARC, DC-CK1, AMAC-1, Ckβ7, | |
| | | MIP-4 |
| CCL19 | ELC, Exodus-3, Ckβ11 | CCR7 |
| CCL20 | LARC, Exodus-1, Ckβ4 | CCR6 |
| CCL21 | SLC, 6Ckine, Exodus-2, Ckβ9, | CCR7 |
| | TCA-4 | |
| CCL22 | MDC, DC/β-CK | CCR4 |
| CCL23 | MPIF-1, Ckβ8, MIP-3, MPIF-1 | CCR1 |
| CCL24 | Eotaxin-2, MPIF-2, Ckβ6 | CCR3 |
| CCL25 | TECK, Ckβ15 | CCR9 |
| CCL26 | Eotaxin-3, MIP-4a, IMAC, TSC-1 | CCR3 |
| CCL27 | CTACK, ILC, Eskine, PESKY, | CCR10 |
| | skinkine | |
| CCL28 | MEC | CCR3, CCR10 |
| CXCL1 | Gro-a, GRO1, NAP-3, KC | CXCR2 |
| CXCL2 | Gro-β, GRO2, MIP-2a | CXCR2 |
| CXCL3 | Gro-?, GRO3, MIP-2β | CXCR2 |
| CXCL4 | PF-4 | CXCR3B |
| CXCL5 | ENA-78 | CXCR2 |
| CXCL6 | GCP-2 | CXCR1, CXCR2 |
| CXCL7 | NAP-2, CTAPIII, β-Ta, PEP | |
| CXCL8 | IL-8, NAP-1, MDNCF, GCP-1 | CXCR1, CXCR2 |
| CXCL9 | MIG, CRG-10 | CXCR3 |
| CXCL10 | IP-10, CRG-2 | CXCR3 |
| CXCL11 | I-TAC, β-R1, IP-9 | CXCR3 |
| CXCL12 | SDF-1, PBSF | CXCR4 |
| CXCL13 | BCA-1, BLC | CXCR5 |
| CXCL14 | BRAK, bolekine | |
| CXCL15 | Lungkine, WECHE | |
| CXCL16 | SRPSOX | CXCR6 |
| CXCL17 | DMC, VCC-1 | |
| XCL1 | Lymphotactin a, SCM-1a, ATAC | XCR1 |
| XCL2 | Lymphotactin β, SCM-1β | XCR1 |
| CX3CL1 | Fractalkine, Neurotactin, ABCD-3 | CX3CR1 |

In some embodiments chemokines showing markedly increased levels of detection and/or presence in BAL samples have been associated with chronic rejection also known as bronchiolitis obliterans (BOS or OB) and/or ACR in subjects following lung transplant. A variety of chemokines are contemplated for the purpose of determining chronic rejection and ACR and include, but are not limited to IL1RA, CXCL10, MCP-1, CXCL9, CXCL11, RANTES, IL-13, IL-17, IL-22, fractalkine, and eotaxin.

In some embodiments, the present teachings provide a method of determining chronic and acute cellular rejection in BAL from a subject. The present teachings can also be used to diagnosis chronic and acute cellular rejection using a BAL sample from a lung transplant recipient. The present teachings are not limited to a particular detection assay. The description below provides non-limiting examples of suitable chemokines, biomolecules, cells and detection methods. The present teachings further provides kits for use in detecting chemokines, biomolecules and cells in BAL. Additionally, kits for the detection of chemokine receptors are also contemplated.

The BAL samples evaluated were from 20 lung transplant recipients who had undergone single or bilateral lung transplant and from whom sequential lung lavage and biopsy specimens had been procured during the first post-transplant year (Example A). Recipients were selected from the McKelvey Lung Transplant Program's (Emory University, Atlanta, Ga.) biorepository of lung transplant recipients prior to any physiologic derangement associated with graft failure.

Recipients had also undergone spirometry (pulmonary function test) testing monthly post-transplant for comparison to pre transplant readings. Recipients are known to normally have optimum post-transplant lung function from 3 to 9 months post-transplant. The majority of recipients are known to loose lung function and a diagnosis of bronchiolitis obliterans syndrome (BOS or OB) is assigned when the forced expiratory volume in 1 second (FEV-1) drops below 20% of peak lung function. Many recipients present with either a decrease in the FEV-1 greater than 10% of peak lung function, but less than 20% of peak lung function prior to being classified as BOS, chronic allograft failure of transplanted lung. Alternatively, some recipients can have a loss in mid-volume expiratory flow that was seen by a loss in the spirometric value FEF25-75. A recipient was classified as having BOS0p (pre-BOS) when they met conditions for either or both of a decrease in FEV-1 and/or a loss in FEF25-75 value. Likewise, the BAL samples were evaluated for infection using standard clinical microbiology testing methods.

In some embodiments a scoring montage was developed to determine the sensitivity and specificity of the use of detection of chemokines in combination and well as in combinations with other biomolecules and/or cells such as CD8 Granzyme B frequency, CD8 GranzymeB mean fluorescence intensity (MFI), CD8 HLA-DR frequency, and CD38 frequency, or at least one cell type selected from eosinophils and basophils. The scoring montage combinations and their respective point systems are illustrated in Table 3, and Example F. In some embodiments the scoring montage was used in a longitudinal analysis of the BAL samples from a recipient in order to determine a course of action, including but not limited to, immunotherapy, immunosuppressive therapy and so on.

In some embodiments the reagents used for detection of the chemokine of interest was affixed to a solid support, including but not limited to a bead, nitrocellulose, a glass slide, a chip, a test strip, and a membrane. The reagents, in some embodiments can be reagents for performing an immunoassay, including but not limited to an ELISA, radioimmunoassay, automated immunoassay, bead assay, and an immunoprecipitation assay. In some embodiments the reagents used are reagents for performing a fluorescently activated cell sorting assay. Such reagents and assays are well known to one of skill in the art and include, but are not limited, e.g., PlexMark™ Biomarker Panel (Catalog no. LHC6007, Invitrogen, Carlsbad, Calif.) and so on.

In some embodiments, a variety of chemokines from Table 1, including but are not limited to IL1RA, CXCL10, MCP-1, CXCL9, CXCL11, RANTES, IL-13, IL-17, IL-22, fractalkine, and eotaxin can also be detected in lung lavage and other body fluids (serum, urine, etc.) using other antibody-based methodologies known to one of skill in the art. In some embodiments, these common assays can be either solution-based or with one or more components attached to a solid phase. The detection of the target material e.g., a chemokine, a chemokine receptor, and the like, can be revealed by a number of different read-out systems where reporting uses visual, chemical, biochemical, mechanical or electronic systems such as those seen in ELISA-like reactions, array-based methods, Western blots, and TagMan® protein ligation assays (PLA).

In some embodiments, detection can be by using ELISA-like assays. In such assays capture antibodies can be bound to a solid phase including, but not limited to, wells of a polystyrene microtiter plate. The capture antibodies can be specific for one ligand and there can be capture antibodies for a variety of ligands, each well having capture antibodies specific for a known ligand. The assay washes away unbound antibody flowed by blocking empty binding sites on the solid phase with inert biochemicals, including, but not limited to, e.g., a protein in each well. The test sample is then incubated in each well and the specific ligand will, if present, attach to the capture antibody. Following a second wash, a secondary antibody is added. The secondary antibody can have a label such as a functional molecule including, but not limited to, an enzymatic marker e.g., alkaline phosphatase or horseradish peroxidase. Excess secondary antibody is then washed away and a substrate is added that exhibits a color change when acted upon by the enzyme. Following incubation, development of a color indicates the presence of the specific ligand in the well. The rapidity of color development or the intensity of color can be correlated with the amount of ligand present, and can be interpreted by comparison with known control wells run in the same assay.

In some embodiments, detection can be by array-based methods as known to one of skill in the art. In array-based methods, the capture antibody can be attached or deposited onto a variety of surfaces types, including but not limited to, e.g., glass, derivatized glass, nitrocellulose, and so on, as an array. One surface can be printed with individual spots containing different antibodies. The surface can have fluids applied to the surface containing the individual antibody spots. The array can then be assayed similar to the ELIS assay described above with the exception that the substrate can be capable of exhibiting visible precipitates on the positive spots (e.g., trimethylbenzidine, (TMB)). TMB is a clear solution that turns into a blue precipitate when oxidized. The detection of the target material by the detection antibody can also be revealed by a number of different read-out systems where reporting uses visual, chemical, biochemical, mechanical or electronic systems. Such system can not positive spots that can be positionally identified, indicating the presence of the specific ligand in the sample. For example, the secondary antibody could be conjugated to a fluorophore (e.g., fluorescein isothiocyanate), and detected by illuminating the array with UV light, and observing the emission at the wavelength appropriate for the fluorophore as would be known to the skilled artisan.

In some embodiments, detection can be by Western blot. In a Western blot the sample can be electrophoresed onto a matrix, blotted onto a matrix, including but not limited to, a filter paper-like matrix, and detected by an antibody bound to a chromophore or fluorophore, as described above. In other embodiments, detection can be y a Protein Ligation Assay. In a Protein Ligation Assay primary and secondary antibodies are conjugated to oligonucleotides. These nucleotides can be designed to form, after enzymatic ligation, a sequence that could be amplified by PCR, as would be known to one of skill in the art. Only molecules that bind both specific antibodies would be physically close enough to ligate and produce a positive PCR reaction. Due to the nature of PCR, this would be a very sensitive test. Detection of a single or a variety of chemokines, chemokine receptors and the like can give an indication of the presence of the ligands being tested as well as quantitation of the amount of the tested ligand in a sample. Identification and detection of a ligand or ligands can be used as indication of the early onset of a graft rejection process such as tissue damage due to inflammation, as disclosed herein.

In some embodiments, the combination of CXCL9 and CXCL10 provided and sensitivity and specificity to determine at least one of risk or increased risk of developing ACR or OB prior to onset of clinically confirmatory criteria such as biopsy or a greater than 20% decrease in FEV-1. In some embodiments, the combination of detecting in a BAL sample CXCL10 at least 15 pg/ml sample and detecting the presence or absence of CXCL9, along with the presence or absence of at least one of IL1-RA and RANTES was strongly indicative of a determination of risk or increased risk of developing ACR or OB prior to clinical presentation.

In some embodiments the present teachings provide a method of determining a treatment course of action for a subject who has undergone a long transplant by reacting reagents for the detection of CXCL10 with a BAL sample from the lung recipient subject and determining the amount of CXCL10 in the BAL sample. In some embodiments a CXCL10 cut-off level of 15 pg/ml in the BAL sample provides a finding that the subject is determined to be at increased risk, risk and/or increased probability of having an episode of acute lung transplant rejection. In some embodiments the finding can be used to determine a treatment course of action for the subject based on the increased probability of acute lung transplant rejection.

In some embodiments, the treatment course of action comprises: the administration of augmented anti-rejection therapy to the subject such as the decision to administer pulse doses of intravenous corticosteriod therapy; continued monitoring of the subject, determining the presence or absence of a concurrent infection by evaluating body temperature of the subject and testing to detect a bacterial, viral or fungal infection. Such administration, monitoring and testing methods are routine and well known to one of skill in the art of transplant rejection.

In other embodiments, the present teachings provide a method of determining chronic and/or acute cellular rejection in a subject comprising contacting a biopsy sample with a reagent for the detection of at least one chemokine receptor such as CXCn, CCRn, CX₃CR1, and XC where n is an integer from 1 to 10. Chemokine receptors are listed in Table 1. In some embodiments the receptor can be detected with reagents for the receptor. In still other embodiments the receptor(s) can be detected by immunoassay or histology analysis. Method of histological analysis are well known to one of skill in the art and can include for example, but not limited to light microscopy, electron microscopy, and historadiography.

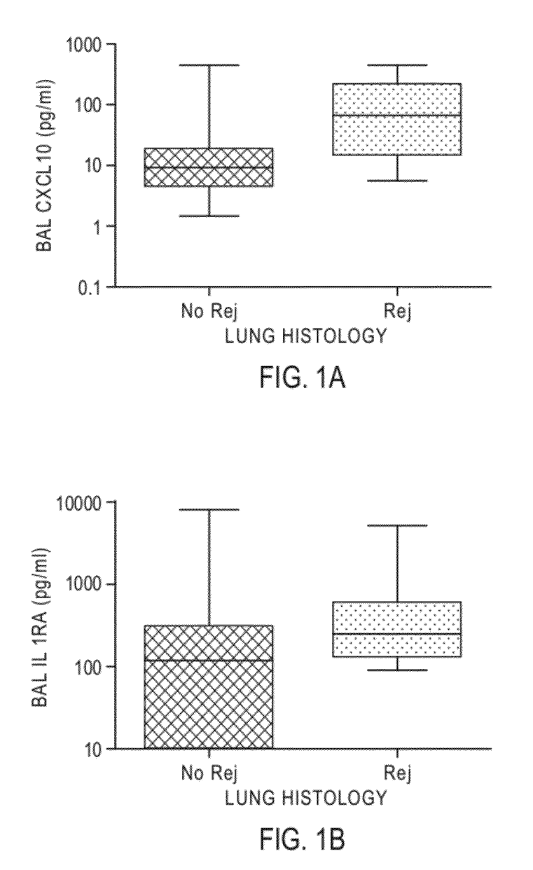

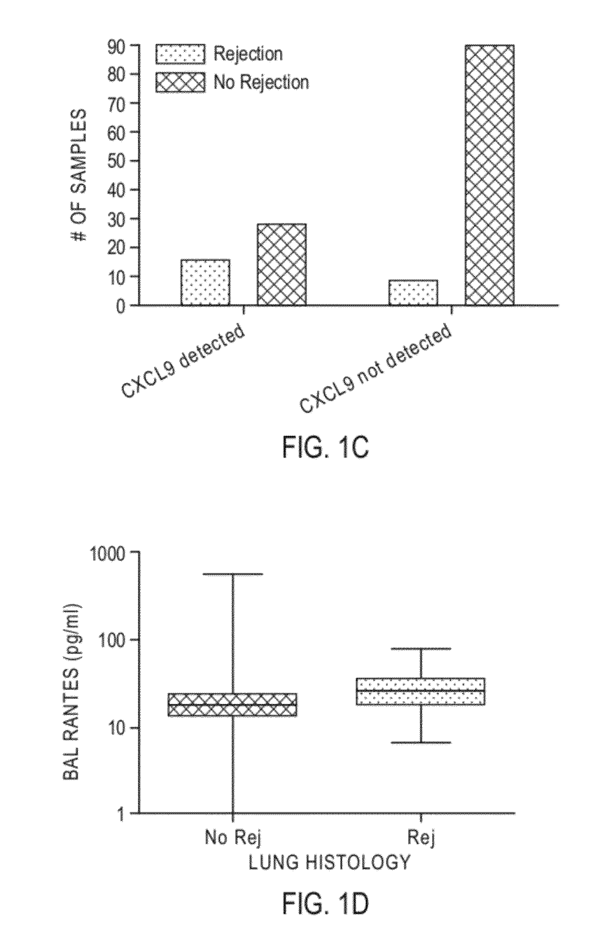

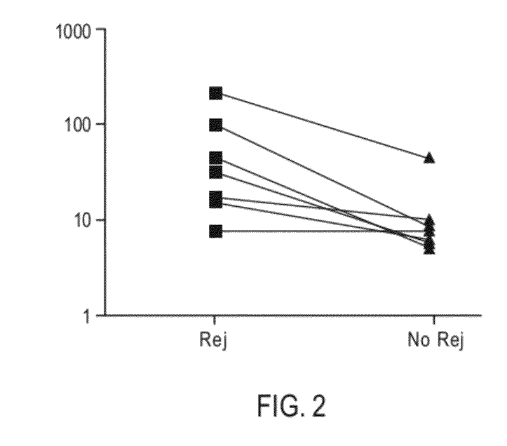

FIGS. 1A, B and C illustrates the detection of CXCL10, IL1RA and RANTES, respectively, in the BAL samples tested for both rejection and non-rejection samples and FIG. 1C illustrates the presence or absence of detection of CXCL9. FIG. 2 illustrates BAL CXCL10 levels during rejection and the decline in CXCL10 levels in patients diagnosed with ACR. The patients with rejection received pulse corticosteroids between the “rejection” and follow-up “non-rejection” biopsy. A significant trend towards decreased BAL CXCL10 levels after rejection is demonstrated. The P value was P=0.03 by the two-tailed paired t-test

In some embodiments, two or more (e.g., 3 or more, 4 or more, etc.) chemokines were detected to provide a risk assessment. The combined presence of any of these markers can provide a more definitive risk assessment of rejection than the analysis of any single marker alone. For example, as illustrated in Table 2, detection of both CXCL10 and CXCL9 together provided higher sensitivity and specificity for ACR compared with each analyte being assessed alone.

Table 2 provides performance characteristics of BAL chemokines in non-paired analysis. Included in this analysis were all BAL samples including samples taken from the first 30 days post-transplant. For the purposes of specificity and sensitivity calculations the following cut off values were used: CXCL9=detected; CXCL10=15 pg/mL, IL1RA=200 pg/mL; RANTES=25 pg/mL).

TABLE 2

| | Sensitivity | Specificity | PPV | NPV | p value | AUC |
|---|---|---|---|---|---|---|
| CXCL9 | 0.65 (0.43-0.84) | 0.76 (0.68-0.84) | 0.35 (0.21-0.51) | 0.92 (0.85-0.96) | 0.0003 | 0.73 |
| CXCL10 | 0.82 (0.61-0.95) | 0.70 (0.61-0.78) | 0.35 (0.23-0.49) | 0.95 (0.88-0.99) | <0.0001 | 0.79 |
| IL1-RA | 0.57 (0.35-0.77) | 0.61 (0.52-0.70) | 0.22 (0.12-0.35) | 0.88 (0.79-0.94) | 0.17 | 0.70 |
| RANTES | 0.43 (0.23-0.66) | 0.84 (0.76-0.90) | 0.35 (0.18-0.54) | 0.88 (0.81-0.94) | 0.009 | 0.71 |
| CXCL9 + CXCL10 | 0.82 (0.48-0.98) | 0.80 (0.69-0.88) | 0.37 (0.19-0.59) | 0.97 (0.89-100) | 0.0001 | — |
| RANTES + CXCL10 | 0.73 (0.39-0.94) | 0.86 (0.76-0.93) | 0.42 (0.20-0.67) | 0.96 (0.88-0.99) | 0.0001 | — |
| IL1-RA + CXCL10 | 0.80 (0.44-0.97) | 0.79 (0.66-0.87) | 0.40 (0.19-0.64) | 0.96 (0.85-100) | 0.0006 | — |
| IL1RA + CXCL9 + CXCL10 | 0.75 (0.35-0.97) | 0.83 (0.70-0.93) | 0.43 (0.18-0.71) | 0.95 (0.84-0.99) | 0.002 | — |

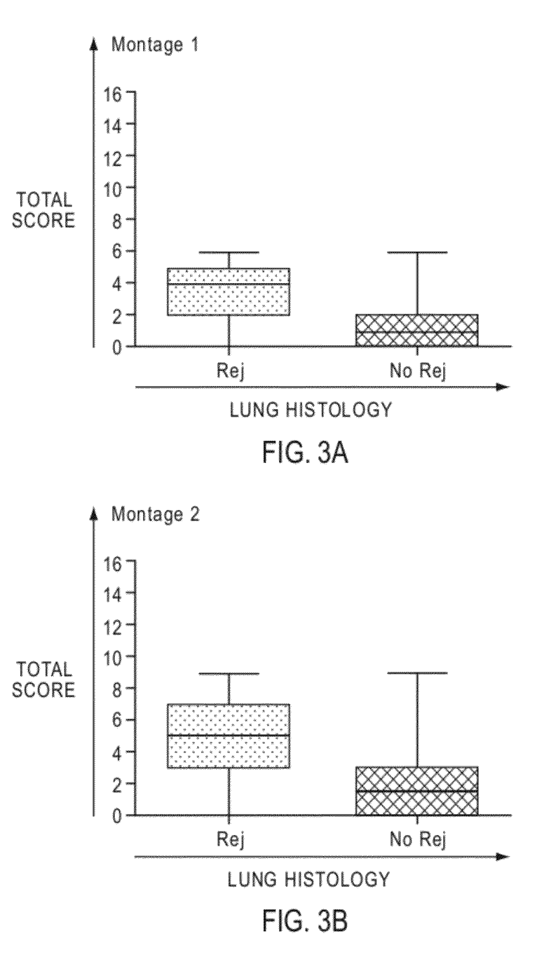

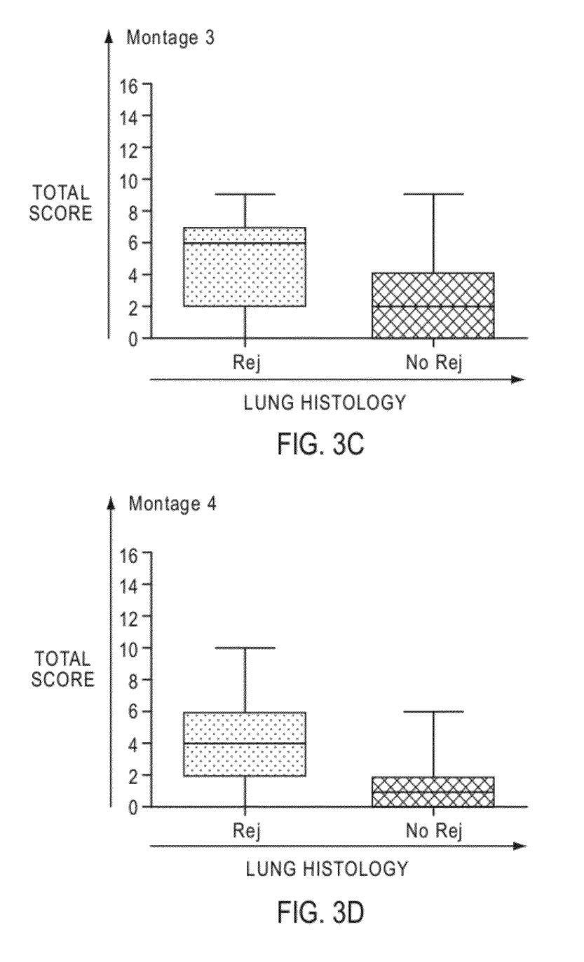

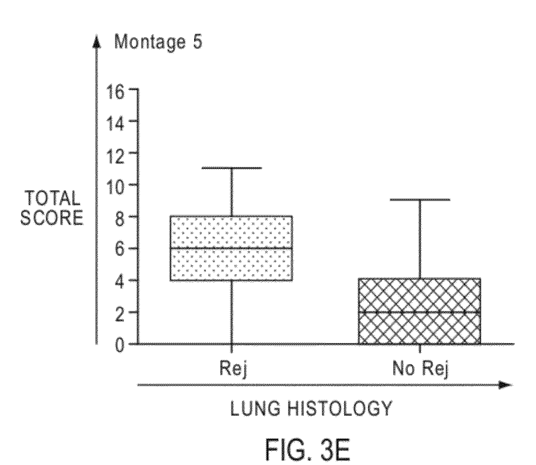

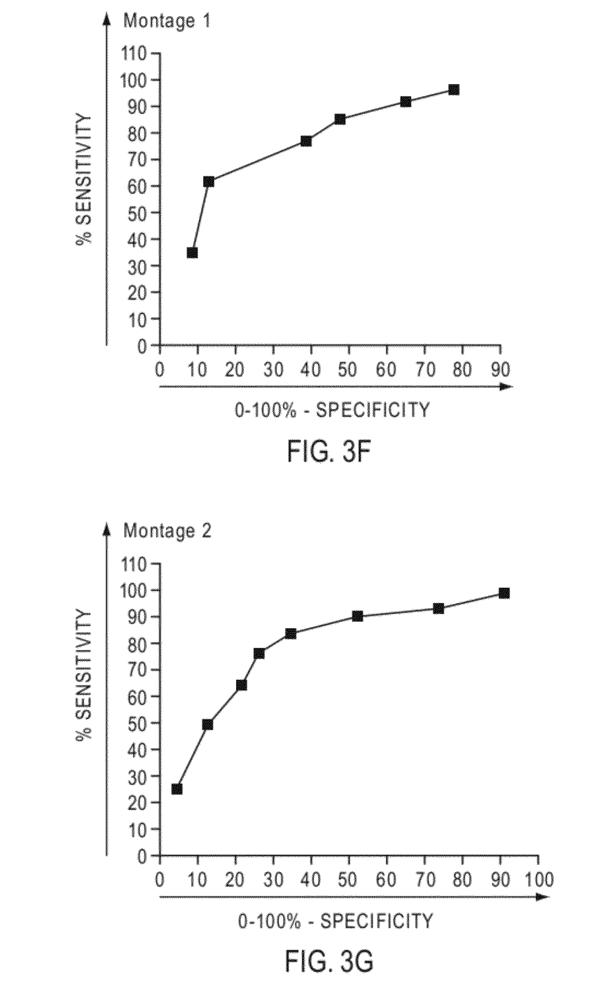

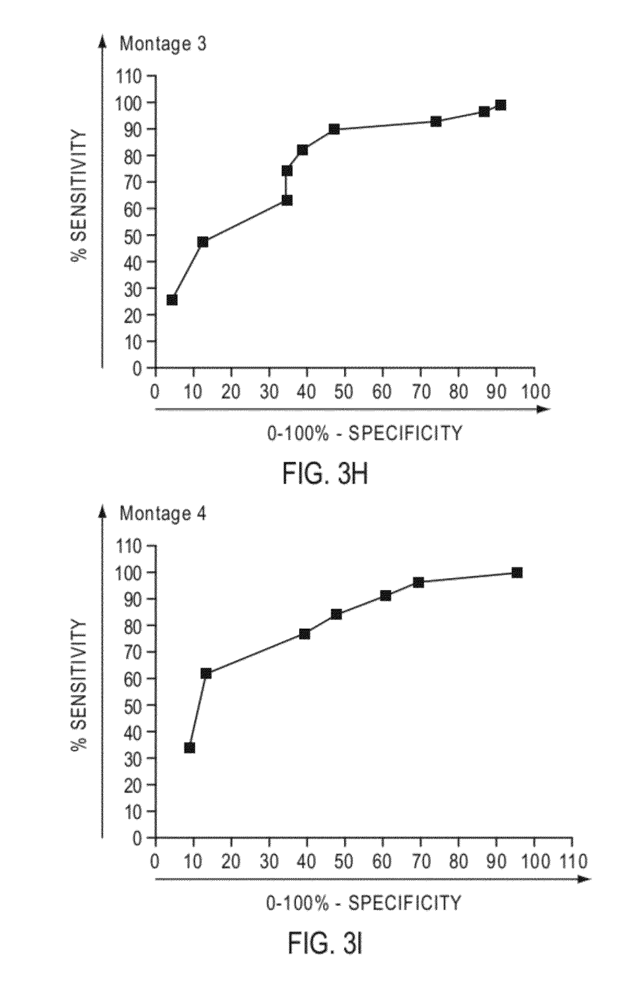

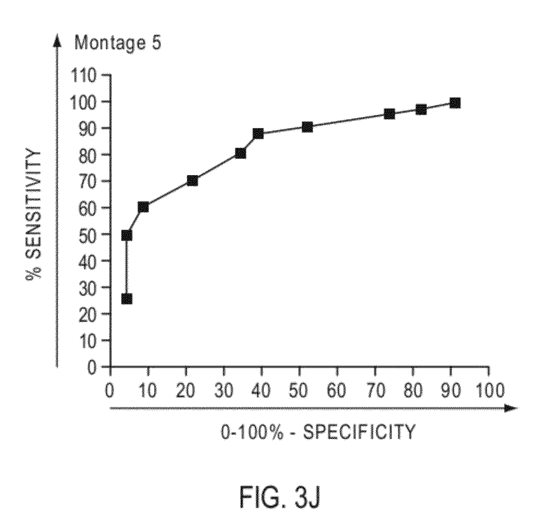

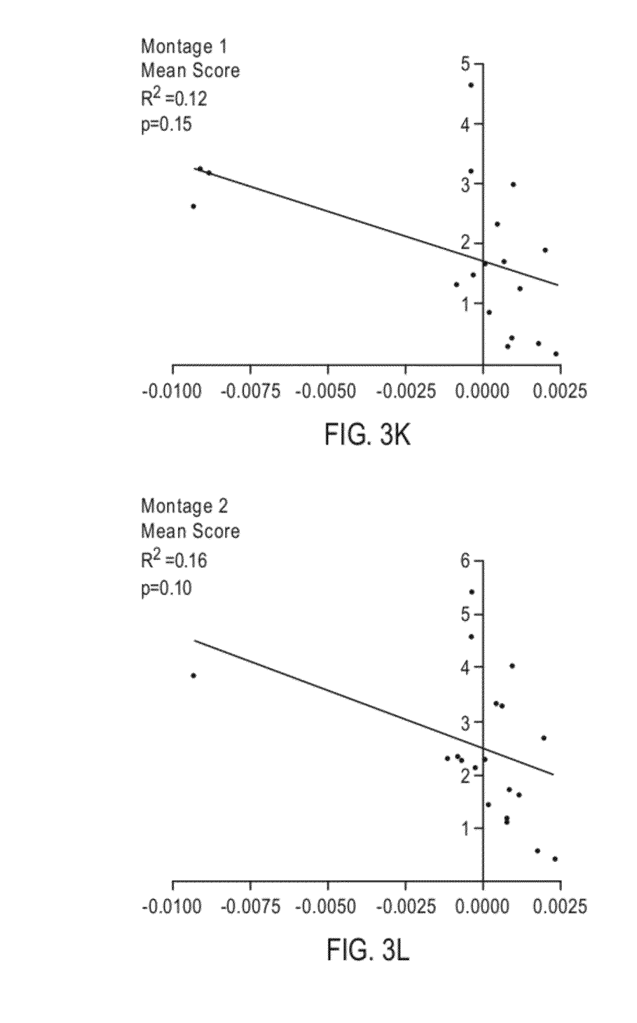

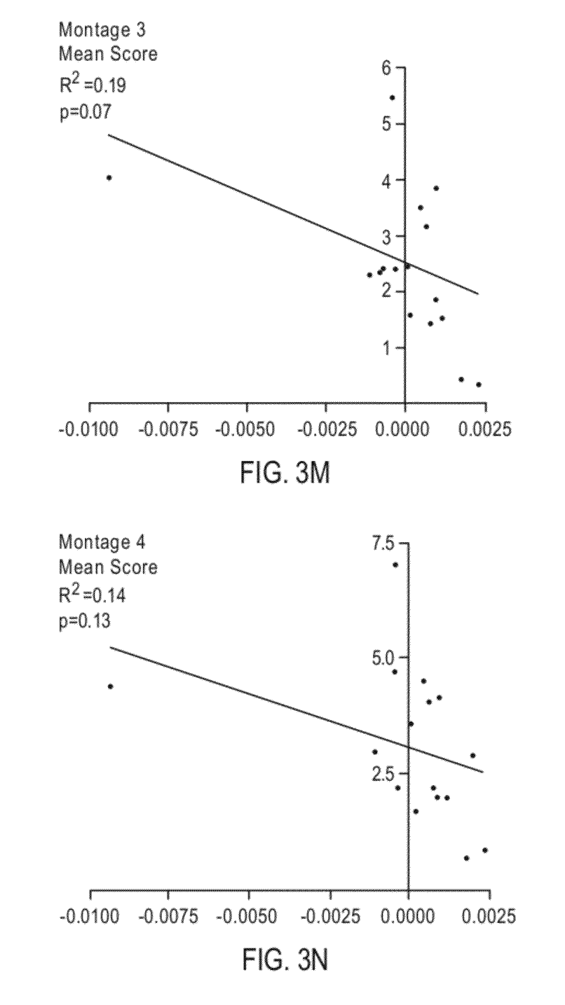

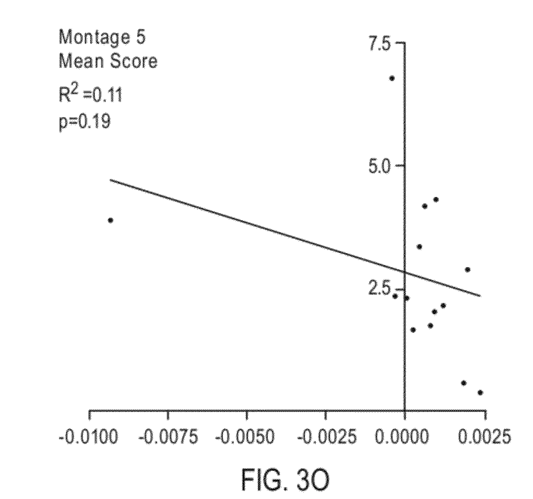

In some embodiments, threshold levels of a particular marker are detected. If the threshold level is reached, risk of chronic rejection or ACR was observed. For example, if 15 pg/ml of the rejection marker CXCL10 in BAL was observed, risk is observed. The present teachings are not limited by the threshold level used in the analysis. In some embodiments, the threshold level was 15 pg/ml or more, 30 pg/ml or more, and 100 pg/ml or more, although both higher and lower threshold values are contemplated for any particular chemokine, as are intervals between these values. FIGS. 3 A-E illustrate a comparison of the five scoring montages between all BAL samples with rejection and those without rejection to detect rejection. P value of <0.0001 by nonparametric t-test for each montage. Example F describes the scoring montage system.

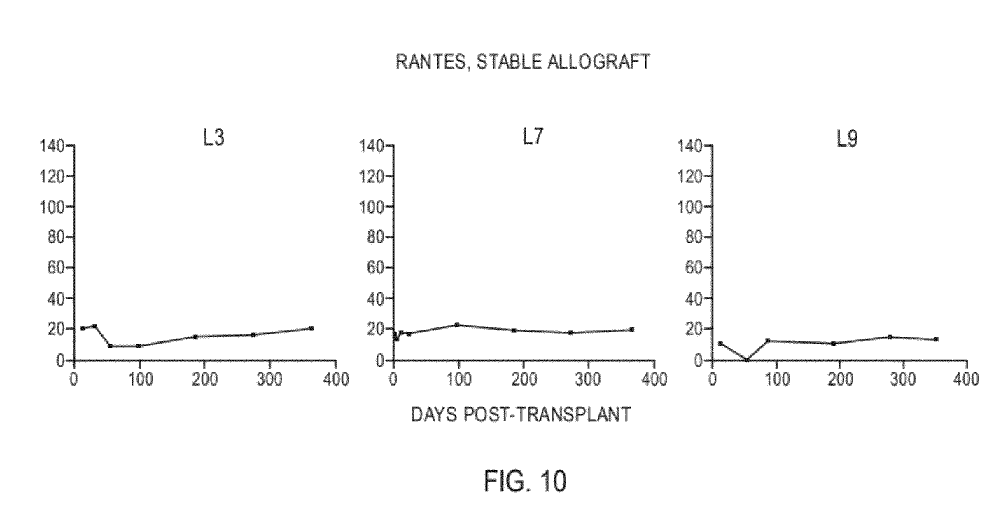

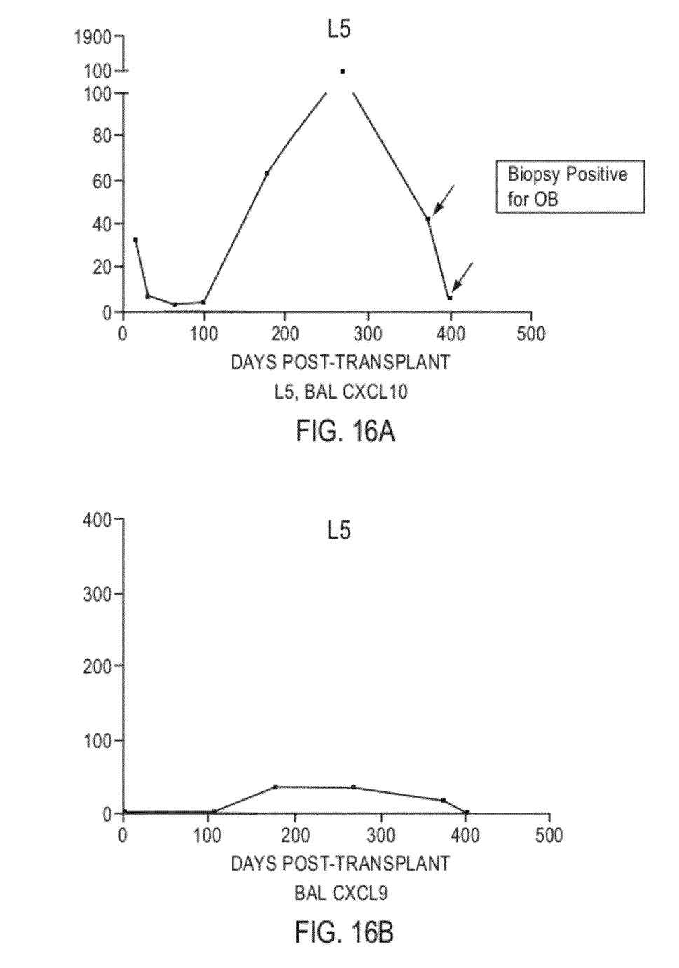

For CXCL-10 there was a striking dynamic range (1.5 pg/mL-443 pg/mL) with the upper limit for detection being roughly 350-450 pg/mL. CXCL10 BAL levels were highly correlative with ACR, p<0.005 (FIG. 1A, Table 2). For IL1-RA a dynamic range was seen ranging from undetected up to 8079 pg/mL. The upper limit of detection for IL1-RA was >50,000 pg/mL. There was a statistically significant increase in IL1-RA in ACR vs. normal samples (FIG. 1B). The vast majority of the samples tested had low levels of CXCL9. In 55/165 samples CXCL9 was detected at or above the level of the lowest standard on the plate (3-5 pg/mL), while it was undetected in 82/165 samples. Notably, CXCL9 was detected in all three samples with OB (FIG. 16B). Based on these findings CXCL9 was analyzed as a dichotomous variable using Fisher's exact test. Detectable CXCL9 was significantly more prevalent in ACR vs. normal samples (FIG. 10). RANTES was detected in all but one sample, with a range of 5 pg/mL-67 pg/mL with one outlier detected at 553 pg/mL. For comparison, the dynamic range of RANTES on the standard curve was 1.87 pg/mL-1102 pg/mL. While the BAL RANTES values had a relatively narrow range, there was a statistically significant difference between ACR and normal samples (FIG. 1D).

The initial analysis compared rejection and non-rejection samples by non-paired analysis and allowed for calculation of specificity and sensitivity performance characteristics. Subsequently the performance of combinations of these analytes was analyzed (Examples E and F). The combination having the best trade off between sensitivity and specificity was CXCL10+CXCL9 (Table 2).

Paired comparison of rejection to non-rejection in paired analysis shows strong effect for CXCL10: The initial analysis did not take into consideration the potential confounding effect of repeated analysis of recipients with continued rejection. Since recipients with ACR could be at an increased probability of subsequent ACR, pooling of all the ACR samples from a particular recipient into the total pool of ACR samples could exaggerate the effect seen. To address this, all recipients who had an episode of ACR followed by a subsequent normal biopsy were compared. There were 7 paired samples available for comparison analysis. Unexpectedly, it was determined that a statistically significant trend was seen for CXCL10, with decreased levels of CXCL10 in the aftermath of rejection (FIG. 2). Results for RANTES, CXCL9, and IL1-RA were not determined to be statistically significant, which can be a reflection of insufficient statistical power.

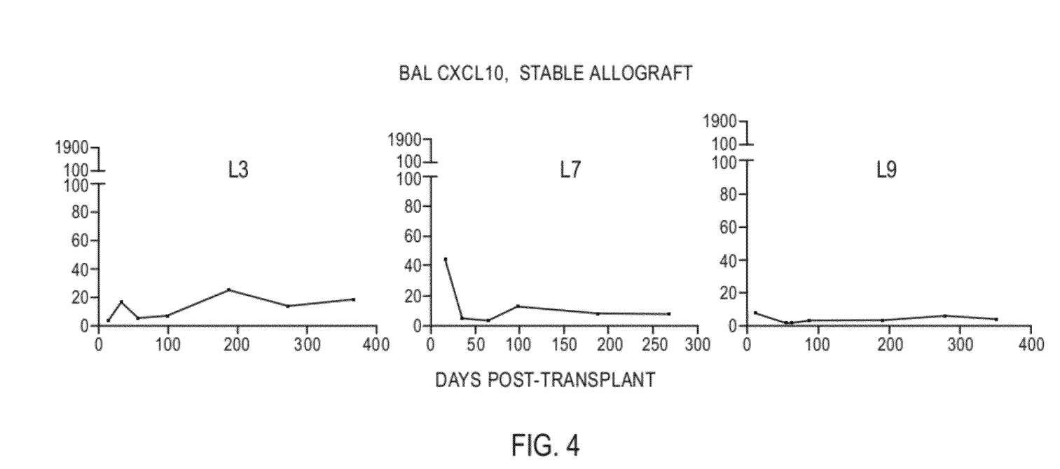

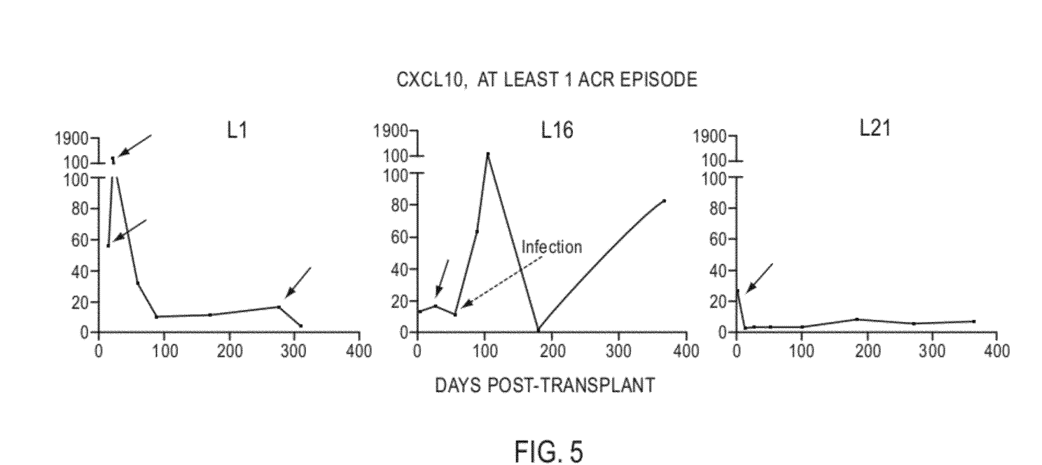

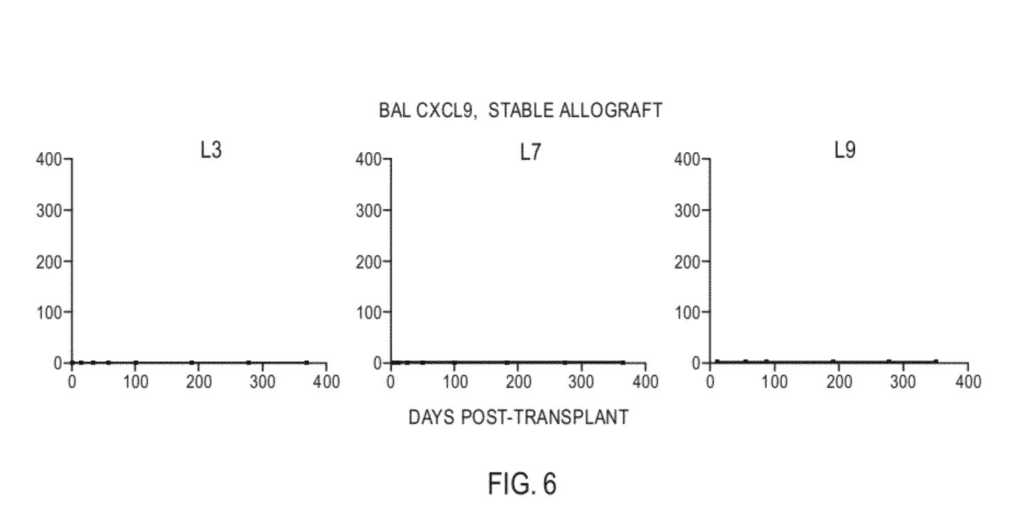

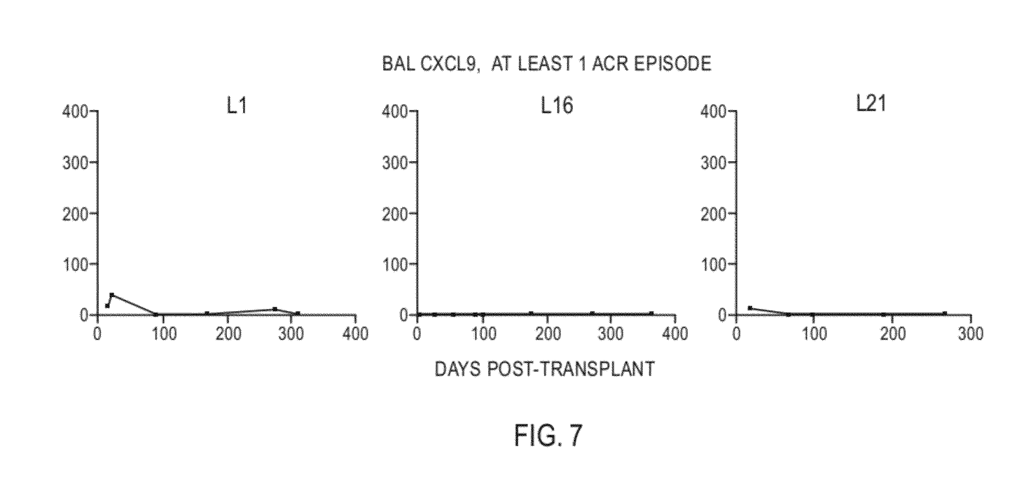

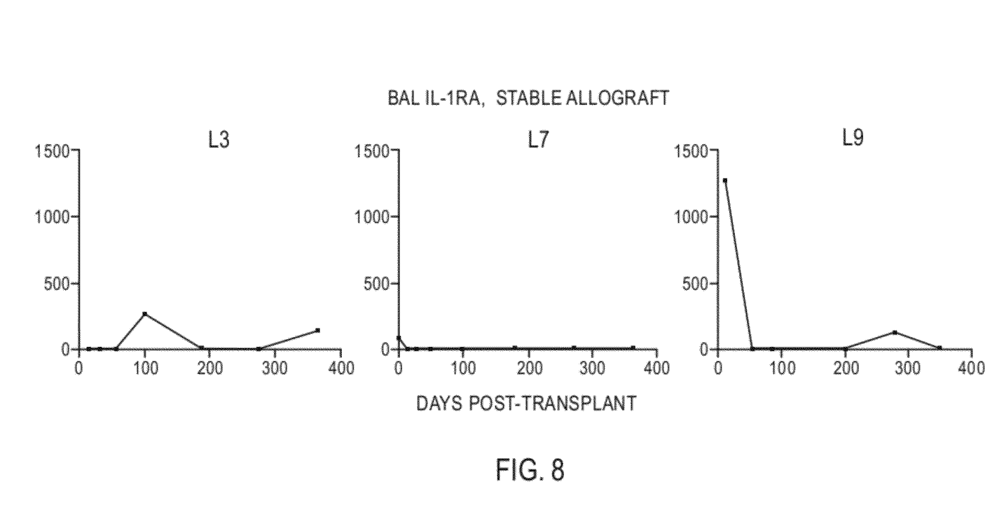

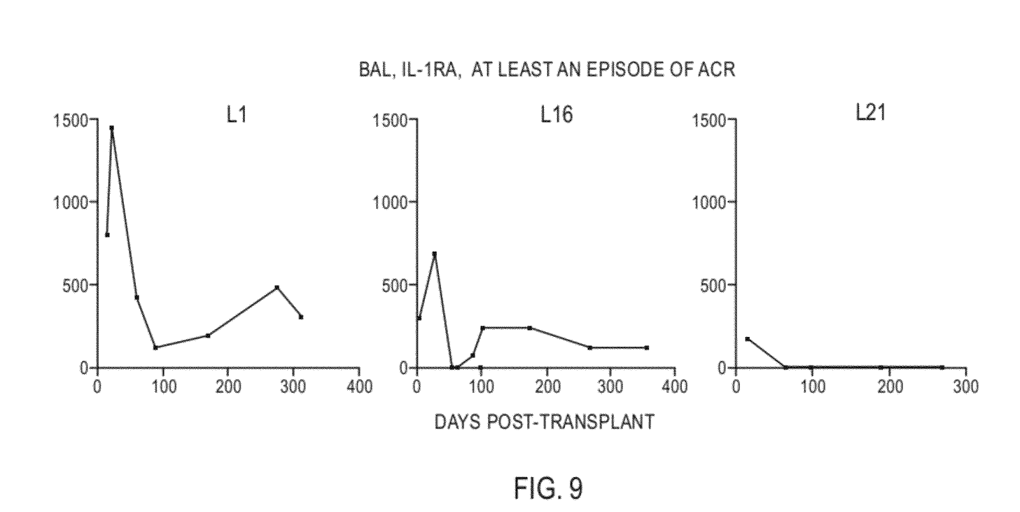

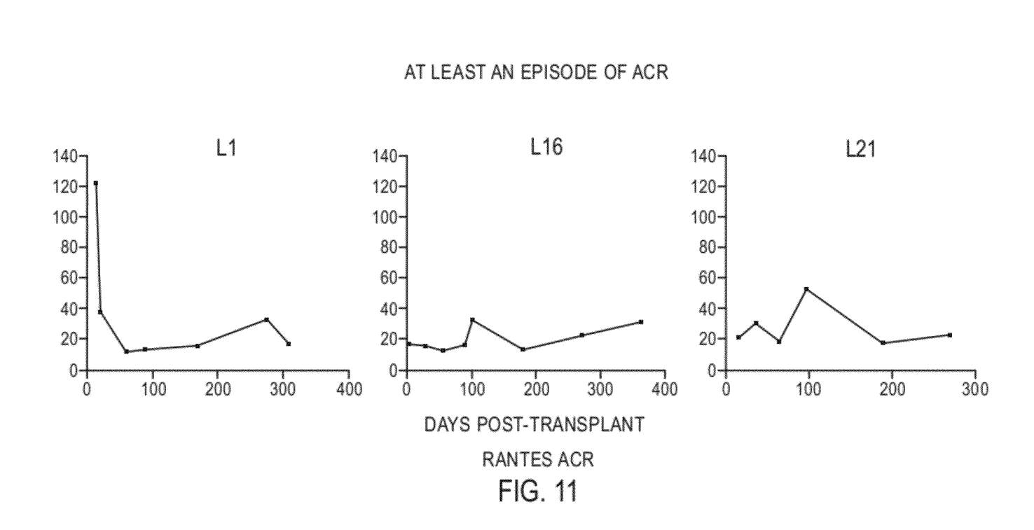

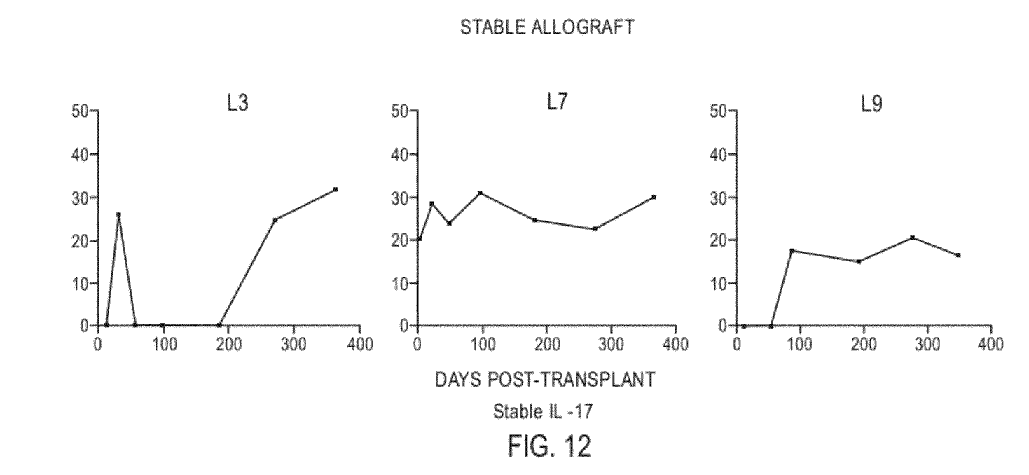

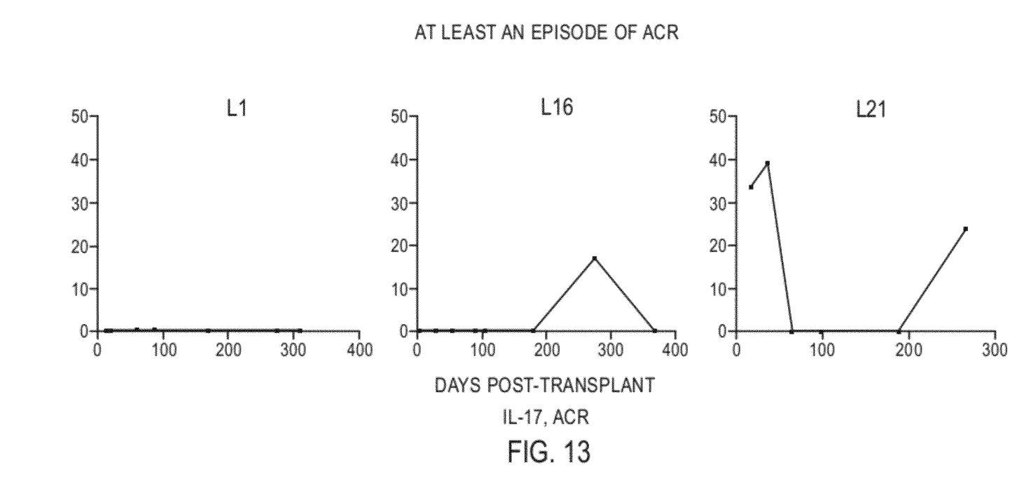

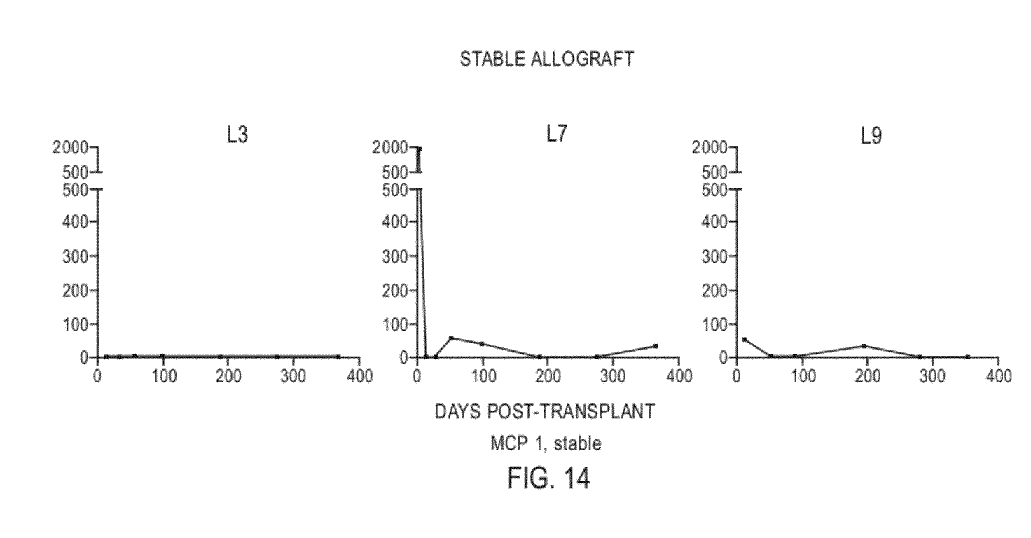

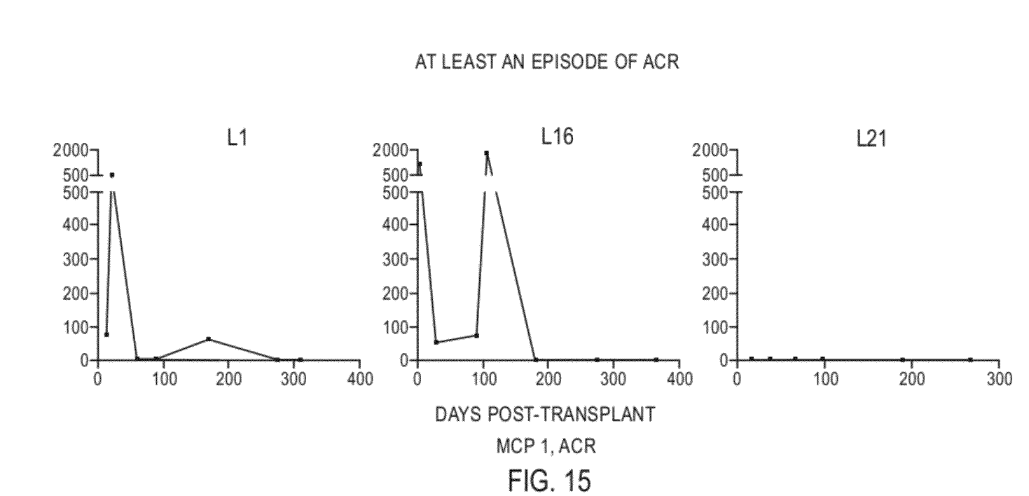

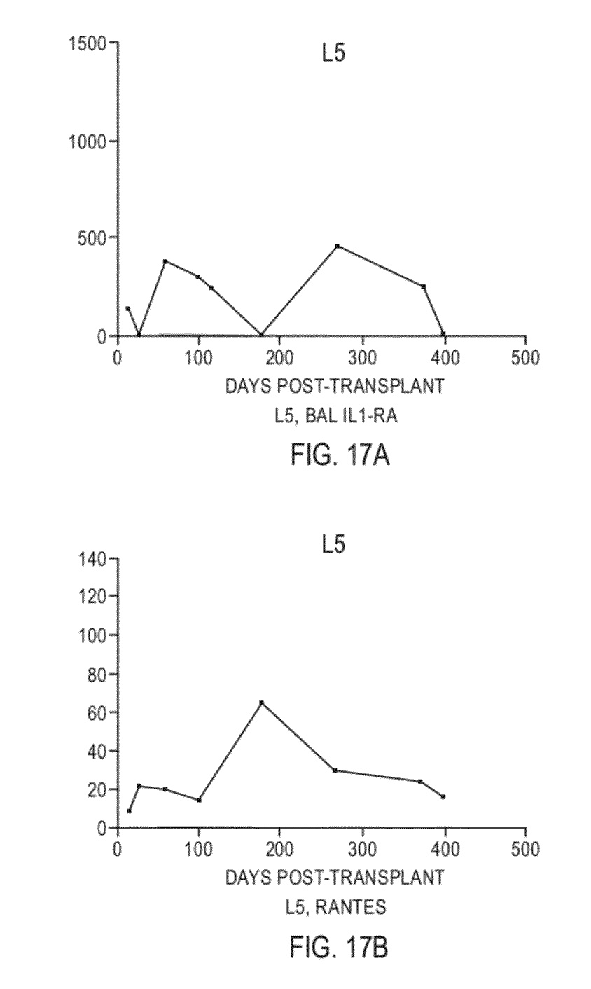

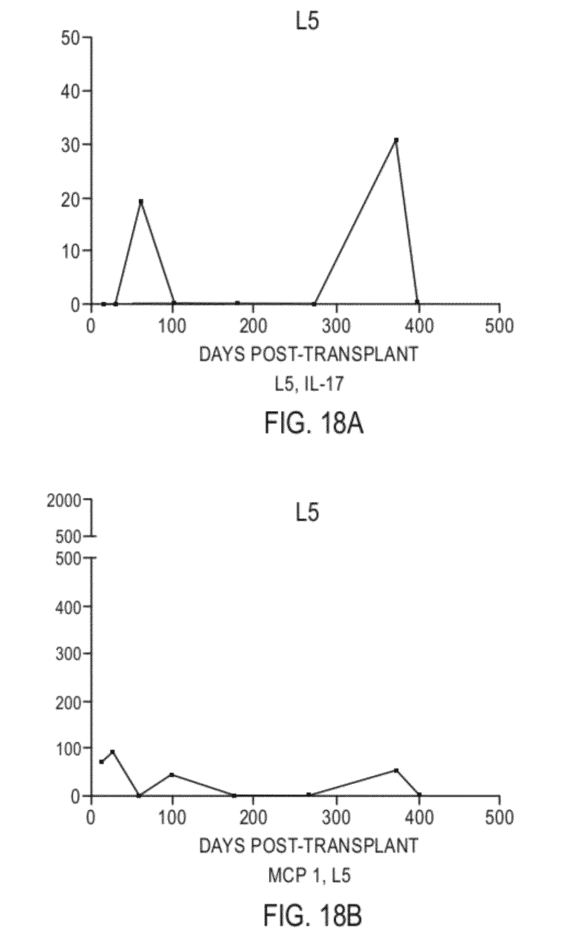

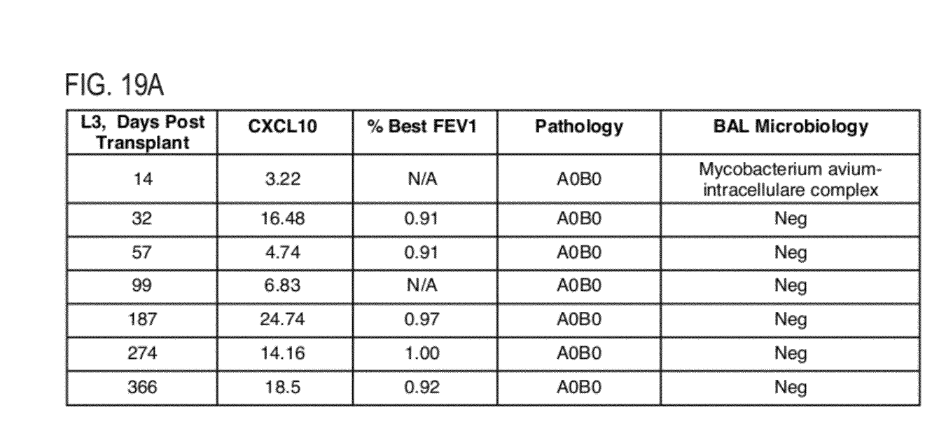

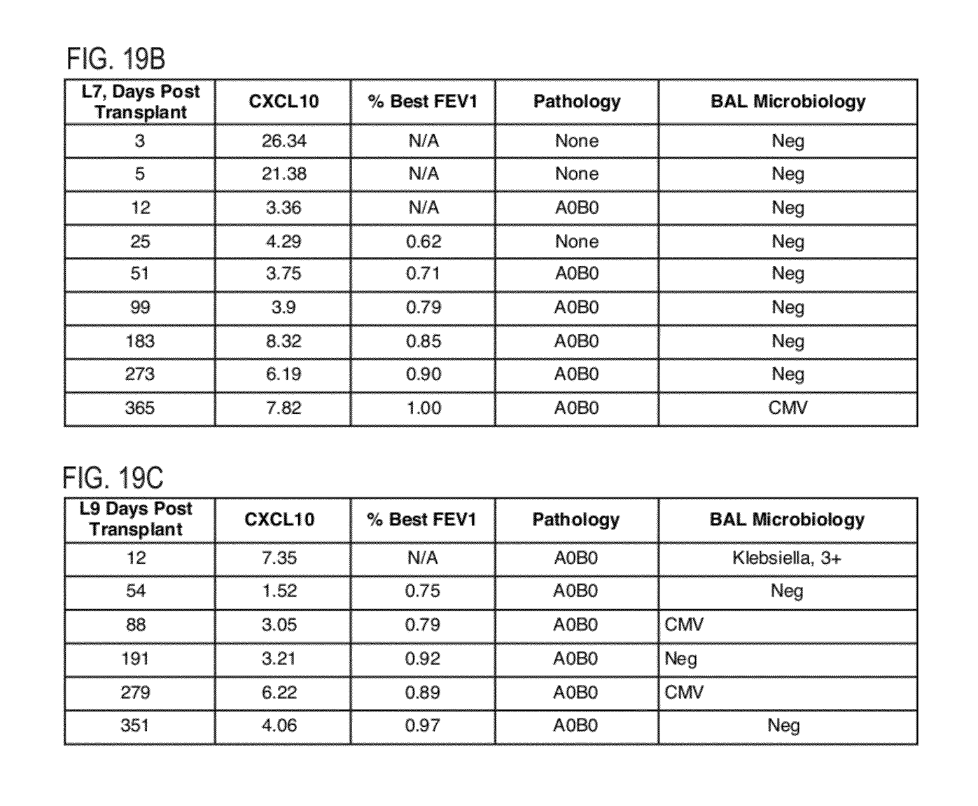

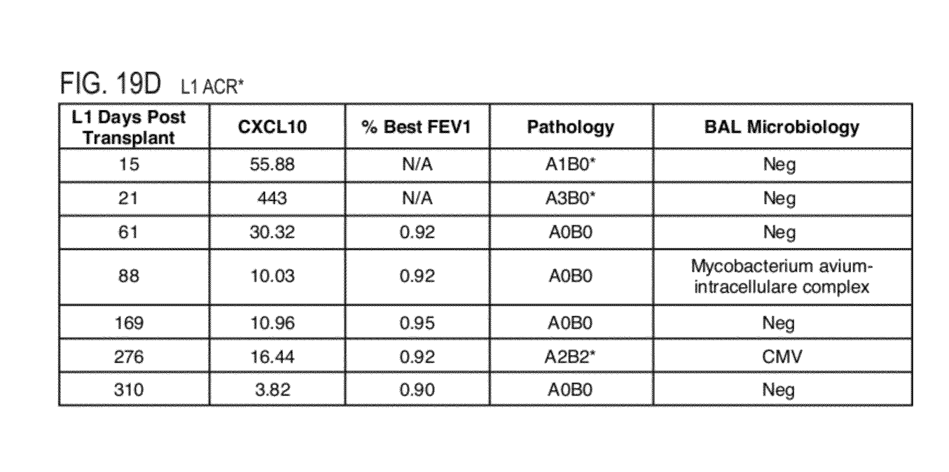

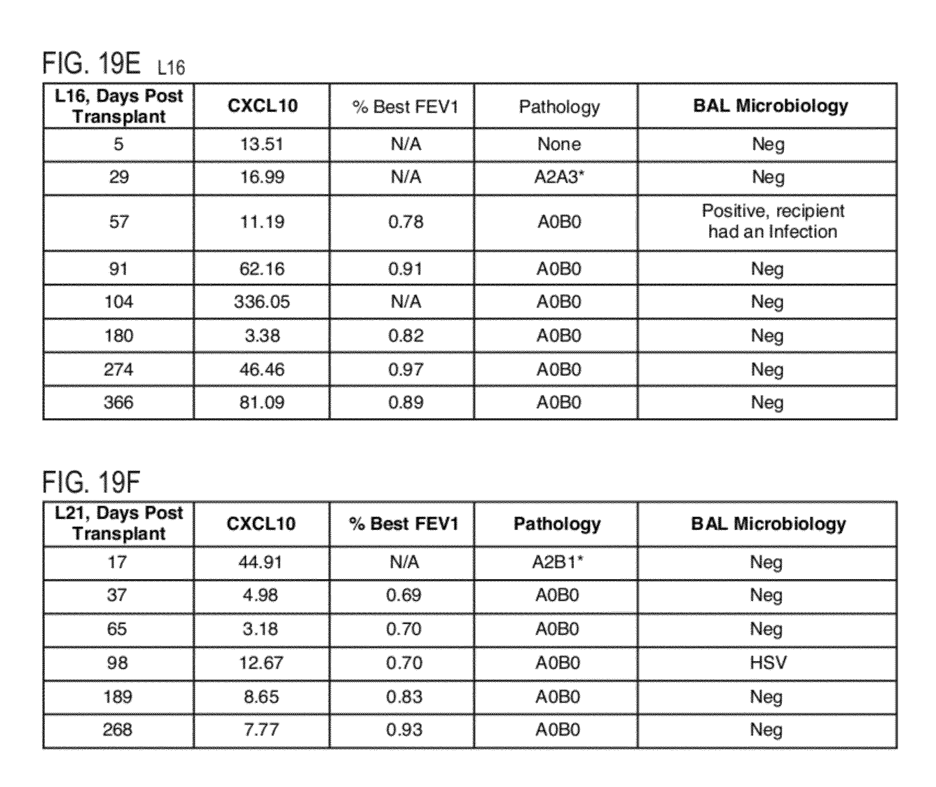

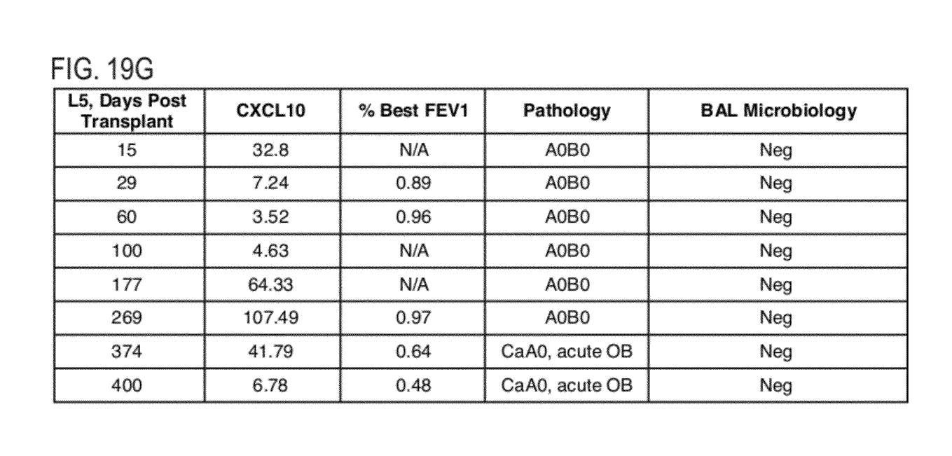

Representative examples of recipients showing no rejection, i.e., a stable allograft are recipients L3, L7 and L9. Recipients L1, L16 and L21 each had at least 1 acute chronic rejection (ACR) episode and recipient L5 developed bronchiolitis obliterans syndrome at 1 year post lung transplant. FIGS. 4-18 show the longitudinal trend of chemokines CXCL10, CXCL9, IR-1RA, RANTES, IL-17 and MCP 1 levels (y-axis, concentration of chemokine in pg/ml) over days post-transplant (x-axis) for sequentially collected BAL samples for each recipient group. FIG. 19A illustrates the CXCL10 levels in sequential BAL samples for the stable allograft recipients. FIG. 19B depicts CXCL10 levels in sequential BAL samples for the ACR representative group.

FIG. 19C illustrates for recipient L5 the unexpected and surprising spike at day 177 post-transplant in CXCL10 levels by comparing sequential BAL samples but prior to a drop in lung function or a pathology finding indicating ACR or BOS. Also surprising is that the graded pathology scoring for rejection was negative (A0B0) at day 177, FEV1 levels were still 97% of normal up to day 269 post-transplant and there was no associated infection that could otherwise account for the presence of a chemokine associated with inflammation. The use of inflammatory markers such as CXCL10 provided the surprising result of being useful in determining rejection in lung transplant recipients and their increased risk for development of BOS prior to a decrease in FEV1 percentage as a measurement of lung function decline. The determination of risk of rejection as a function of chemokine levels such as CXCL10, CXCL9, IR-1RA and MCP 1 can be used to adjust immunosuppressant therapies or take intervention steps such as a pulse of steroids, including but not limited to, 3 days of pulse corticosteroids.

The extensive analysis of sequential samples taken from BALF collected in the first year post lung transplant revealed a phenotype which both correlated with the establishment of peak lung function and predicted patients at risk of death and chronic rejection. It can be suggested that in patients at risk for graft loss the IFNG dependent chemokines CXCL9 and CXCL10 will be found to be elevated in the BALF of these patients. These data were consistent with previously published data showing that these chemokines were elevated in patients with already established BOS and in animal models of chronic rejection (Agostini et al, 2001, and Belperio, 2002, 2003). It can be suggested that these findings lay the groundwork for clinical studies which attempt to utilize these markers in real-time to make clinical decisions. For example, patients found to have elevations of either or both CXCL9 and CXCL10 may be candidates for augmented immunosuppression. Further, patients with repetitive low levels of these chemokines may be candidates for targeted de-escalation of immunosuppression. The use of an AUC analysis to approximate cumulative exposure over time and to give weight to persistent as opposed to transient inflammatory activity is suggested by the study. The study also suggests the feasibility of using repetitive BALF sample analysis to make clinical decisions that impact future lung function based on the extent to which CXCL9 and CXCL10 are elevated before lung function starts to decline. While only 10 episodes of graft failure were observed, 9 of 10 patients displayed significant elevations of CXCL10 from 3-9 months prior to death or retransplantation. These data are consistent with BO deriving from the cumulative effects of chronic inflammatory activity.

While it is noteworthy that CXCL9, CXCL10 and to a lesser extent MCP-1 BALF levels tracked with relevant clinical readouts post lung transplant, it is also remarkable that several other biomarkers did not appear to correlate with meaningful outcomes. A significant relationship between graft failure, BOS, acute rejection, or lung improvement post-transplant with respect to IL-13, IL-17, RANTES or IL1-RA was not seen. For the case of IL-17, there is growing data linking cellular Th17 responses driven by IL-17 to the subsequent development of BOS (Burlingham W J, Love R B, Jankowska-Gan E, et al. “IL-17-dependent cellular immunity to collagen type V predisposes to obliterative bronchiolitis in human lung transplants,” J Clin Invest 2007; 117:3498-506.). Further, it is notable that Vanaudenaerde et al. have demonstrated elevated BALF protein levels in patients with acute rejection and BOS compared to stable patients (Vanaudenaerde B M, De Vleeschauwer S I, Vos R, et al. “The role of the IL23/IL17 axis in bronchiolitis obliterans syndrome after lung transplantation,” Am J Transplant 2008; 8:1911-20 and Vanaudenaerde B M, Dupont L J, Wuyts W A, et al. “The role of interleukin-17 during acute rejection after lung transplantation,” Eur Respir J 2006; 27:779-87). In those studies, the mean IL17 concentration in the BALF in the setting of ACR and BOS was 3 and 2 pg/mL respectively. With the multiplex ELISA platform utilized here the lower limit of detection of IL-17 was 10 pg/mL. Hence, a significant number of samples were classified as not detected and this may have limited the ability to draw meaningful conclusions with respect to IL-17. IL1-RA represented an attractive potential biomarker as it demonstrated the most significant dynamic range of all the markers tested, prior work has demonstrated an inverse relationship between IL1-RA and renal function in kidney transplantation (Sadeghi M, Daniel V, Naujokat C, et al. “Decreasing plasma soluble IL-1 receptor antagonist and increasing monocyte activation early post-transplant may be involved in pathogenesis of delayed graft function in renal transplant recipients,” Clin Transplant; 24:415-23), and drug therapy harnessing the anti-inflammatory promise of IL1-RA has been demonstrated in rheumatoid arthritis (Mertens M, Singh J A. “Anakinra for rheumatoid arthritis: A systematic review,” J Rheumatol 2009; 36:1118-25). In spite of the relative detectability of BALF IL-1RA, its presence did not correlate with outcome in our study. This may reflect the fact that IL-1RA may be elevated in both the setting of inflammation as a counter regulatory cytokine in patients experiencing rejection and also may exist at high basal levels in patients with prolonged quiescence. Hence, our analytic approach may not have been sensitive enough to detect an effect of IL1RA even if one exits.

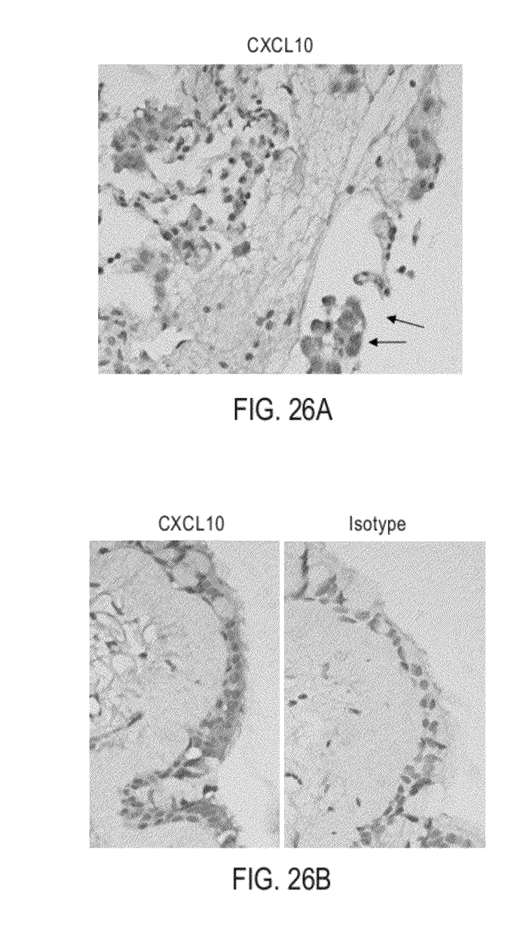

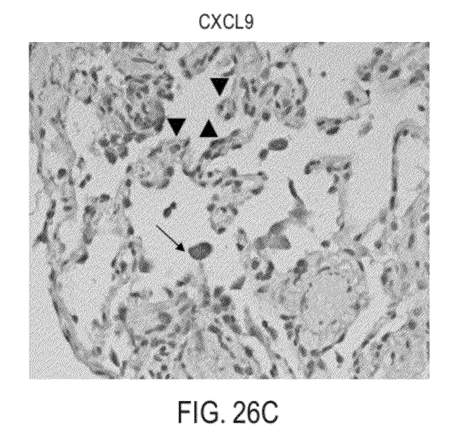

A central question to any clinical study utilizing biomarkers to assess a relevant clinical endpoint is the extent to which the markers studied are actually involved in the disease process. For the case of CXCL9 and CXCL10, it is plausible that these IFNG dependent chemokines are relevant for progression of BOS. For example, the histologic examination of transbronchial lung biopsy specimens in patients with very high levels of CXCL9 and CXCL10 demonstrated strong staining of the bronchiolar epithelium for these markers (FIGS. 26A-26C). FIG. 26A shows positive staining for CXCL10 of alveolar macrophages. FIG. 26B shows positive staining of bronchiolar epithelium compared to isotype control antibody. FIG. 26C shows CXCL9 staining of both alveolar macrophages (arrows) and the alveolar epithelium (arrowhead). Chronic rejection in lung transplant is characterized by a conversion of the bronchiolar lumen from a columnar epithelium to a stratified layer of fibroblastic tissue. Hence, the finding that the relevant pre-BO biomarker is found within the tissue compartment of interest adds to the biologic plausibility that these IFNG-dependent chemokines are actually participating in the disease process. CXCL9 and CXCL10 are chemoattractive for T cells expressing CXCR3. Therefore, one potential mechanism involved in the pathogenesis of BO is that these chemokines are attracting relevant T cells into the graft that bare the cognate receptor and can cause graft injury. Indeed, a growing body of work suggests that lymphocytic bronchitis (characterized by lymphocytes invading the bronchiolar wall) is a precursor toward BO (Glanville A R, Aboyoun C L, Havryk A, Plit M, Rainer S, Malouf M A. “Severity of lymphocytic bronchiolitis predicts long-term outcome after lung transplantation,” Am J Respir Crit Care Med 2008; 177:1033-40). Future studies to determine if elevations of CXCL9 and CXCL10 are linked with concurrent increases in CD4 and CD8 T cells that are CXCR3+ will be undertaken. The present study has shown that persistently elevated levels of CXCL9 and CXCL10 are strongly associated with graft and patient survival, and herald the onset of deteriorating lung function in human lung transplant recipients. These data support the design and conduct of a prospective clinical trial investigating the use of BALF chemokine assessment as a tool to guide clinical immune management.

The present teachings are also directed to kits for detection of chemokines in biological samples that utilize the methods described above. In some embodiments, a basic kit can comprise a reagent for the detection of CXCL10 and at least one analyte selected from of IL1RA, MCP-1, CXCL9, RANTES, IL-13, IL-17, IL-22, fractalkine, and eotaxin. The kit can also optionally contain instructions for use. A kit can also have other optional kit components, such as, for example, one or more of a 96-well filter plate, wash solution(s), buffer, and reagents used in an immunoassay or a fluorescently activated cell sorting assay or an ELISA assay as well as reagents for detecting biomarkers selected from CD8 Granzyme B, CD8 GranzymeB MF1, CD8 HLA-DR, and CD38, and/or for detection of a cell type from the group consisting of eosinophils and basophils. In some embodiments, the biomarkers can be detected by flow cytometry, histology and other methods known to one of skill in the art. The amounts of the various reagents in the kits also can be varied depending upon a number of factors, such as the optimum sensitivity of the process. It is within the scope of these teachings to provide test kits for use in manual applications or test kits for use with automated sample preparation, reaction set-up, detectors or analyzers.

### Examples

### A. Recipient Selection and Sampling Protocol

20 lung transplant recipients were identified who had undergone single or bilateral lung transplant and for whom sequential lung lavage specimens had been collected during the first post-transplant year. Surveillance bronchoscopy was performed on each recipient at 2 weeks, 1, 2, 3, 6, 9, and 12 months post-transplant according to the McKelvey Lung Transplantation surveillance transbronchial biopsy protocol- (2006, Emory University). During the bronchoscopy, 6-10 pieces of alveolated lung tissue was obtained and scored for rejection by a pulmonary pathologist. Rejection was graded from 0-4 in both venuoles (designated by A) and bronchioles (designated as B) by criteria established by the International Society of Heart and Lung Transplantation. A score of zero indicated no rejection. Scores of 1-4 indicated progressive worsening of rejection: 0 as none, 1 as minimal, 2 as mild, 3 moderate, and 4 as severe. Any rejection score was scored as being positive for rejection. Because of recipient factors and clinical judgment, not all recipients underwent exactly seven bronchocopies with lung biopsy in the first year. Additionally, some recipients underwent bronchoscopies at time points that were out of cycle with the clinical protocol, such as in the setting of suspicion of acute rejection. As a result, for the recipients studied there were between 5 and 10 total bronchoscopies for analysis.

### Patients:

The Institutional Review Board at Emory University approved this study. We assessed all lung transplant recipients transplanted at Emory University Hospital between 2006 and 2008 for inclusion into this study. Patients accrued to this study were required to have at least one year of post-transplant survival and at least 4 bronchoscopies that were obtained in the first post-transplant year for which BAL fluid was available for analysis. Patients enrolled agreed to allow us to perform assays on BALF which was left over from routine protocol or for cause bronchoscopies. Of the 53 lung transplants performed during this time period, 40 patients met these criteria and were included in this analysis. Of the 40 patients assessed, there were 6 single lung transplants and 34 bilateral transplants. 22 patients were classified as having obstructive lung disease prior to transplant with disease processes that included emphysema (19 patients), cystic fibrosis (1 patient), bronchiectasis (1 patient) and sarcoidosis (1 patient). 18 patients were classified as having restrictive lung disease prior to transplant with diseases that included idiopathic pulmonary fibrosis (15 patients), acute interstitial pneumonia (1 patient), hypersensitivity pneumonitis (1 patient) and silicosis (1 patient). All patients characterized as having obstructive disease had FEV1/FVC ratios less than 30. All patients characterized as having restrictive disease had a total lung capacity of less than 80% of predicted. All patients were treated with the same initial immunosuppressive therapy which included induction therapy with basiliximab and maintenance immunosuppression with prednisone, tacrolimus and azathioprine. Following transplantation each patient had at least monthly pulmonary function testing performed during which the Forced Vital Capacity (FVC) and the Forced Expiratory Volume in 1 second (FEV1) was calculated.

### B. Bronchoalveolar Lavage Sample Collection

Bronchoscopy was performed and bronchoalveolar lavage fluid (BAL) was obtained by wedging the bronchoscope into the right middle lobe of left lingula. Sequential aliquots of normal saline in 6×30 mL increments were instilled into the wedged segment, followed by aspiration of the fluid. In general, fluid recovery was greater that 50% volume instilled. Lavage fluid was centrifuged at 1300 rpm for 5 minutes. The supernatant fraction of this fluid was aspirated from the cellular fraction and frozen at −80 C until analyzed using a Bio-Plex 200 (Luminex) (BioRad, Hercules, Calif.).

### CXCL9/CXCL10 Study:

Surveillance bronchoscopy was performed at predetermined time-points post-transplant: 2 weeks, 1 month, 2 months, 3 months, 6 months, 9 months, and 12 months. Not all patients had bronchoscopy performed at each time point at the clinical discretion of the treating pulmonologist. Further, in the aftermath of histologic rejection some patients received follow-up bronchoscopy outside of the surveillance time points. Finally, not every bronchoscopy yielded sufficient extra fluid for our analysis. In total, from 40 patients analyzed over the first year post-transplant we performed multiplex ELISA on 290 separate samples. Rejection was graded on transbronchial lung biopsy pieces obtained during bronchoscopy by a dedicated pulmonary pathologist in 270 of the 290 samples analyzed.

BALF was obtained during routine bronchoscopy by wedging the bronchoscope into either the right middle lobe or lingular bronchus. A total of 5, 30 mL aliquots of normal saline were instilled into the lung and lavage fluid were obtained by suctioning. Fluid return was generally 70-100 mL total. BALF was immediately centrifuged at 1300 rpm×5 minutes and the supernatant portion was removed and frozen at −80 C until analyzed by ELISA.

The cellular fraction was assessed by flow cytometry. Cells were labeled in various panels with fluorescent antibodies to CD3 PerCPCy5.5 (Becton Dickenson), CD3-Alexa Flour 700 (BD Pharmingen clone UCHT1), CD4-Pacific Blue (BD Pharmingen clone RPA-t4), CD8-Pacific Orange (Invitrogen), HLADR-PECY7 (BD Pharmingen), CD38-PerCPCy5.5 (BD Pharmingen) and CXCR3-APC (BD Pharmingen), and GranzymeB Alexa Flour700 (BD Pharmingen). All labeling was carried out in the dark in PBS+2% fetal bovine serum. For the case of Granzyme B, cells were permeablized with fix/perm buffer (BD Pharmingen) prior to labeling. Cells were analyzed on an LSRII flow cytometer (Becton Dickenson) and data was analyzed using FloJo software (TreeStar, Ashland, Oreg.). To quantify the frequency of GranzymeB hi cells, first light scatter gates were used to identify lymphocytes. Next CD3+CD4−CD8+ events were gated. GranzymeB hi cells were defined as having a fluorescence greater than 1000 out of a possible total channel fluorescence of 10,000 because 1000 defined a clear population of granzymeB hi cells when peripheral blood lymphocytes were analyzed. The GranzymeB hi frequency was the percentage of all CD3+CD4−CD8+ cells that were GranzymeB positive. To calculate the frequency of HLADRhi CD38hi CD8 cells the total population of CD3+CD4−CD8+ cells were gated and assessed as the percentage of that population which was dual positive for HLADR and CD38. In some recipients the frequency of CXCR3+CD8 cells was calculated. This was done by gating on the CD3+CD4−CD8+ population and measuring the percentage of cells with a CXCR3 fluorescence of greater than 100. In order to ensure equal comparisons of samples taken at different times, the cytometer was calibrated using mid-range calibration beads prior to data acquisition. There was an inverse relationship of CXCR3 levels on BAL T cells verses GranzymeB levels.

### C. Chemokine Analysis

The BAL supernatant was assessed for the presence of seven chemokines: CXCL10, CXCL9, IL1-RA, RANTES, IL-13, IL-17, and MCP-1 for analysis of acute rejection using the PlexMark™ 3-A Human Biomarker Panel (Cat. No. LHC6007; IL1-RA Cat. No. LHC0711; MCP-1 Cat. No. LHC1011; RANTES Cat No. LHC1031; and IL-17, Cat. No. LHC0171, Invitrogen, Carlsbad, Calif.). The assays were done according to the manufacture's protocol (Invitrogen, P/N PRLHC6007), incorporated by reference herein. The BAL chemokines were assessed using a custom 96-well plate from Invitrogen. All samples were thawed on the day of analysis and run in duplicate. For each recipient, an entire years worth of BAL was run on the same plate. Concentrations of each of the chemokines were established using regression from a standard curve. FIGS. 4-18 are graphical depictions of the levels of each chemokine and FIGS. 19A-C are the results of CXCL10 along with corresponding results for % FEV-1, Pathology grading of biopsy samples and Microbiology results of the BAL samples. CXCL9/CXCL10 Study:

Custom Multiplex Bead Kits were obtained from Invitrogen Corporation (Carlsbad Calif.). Capture beads with unique bead regions suitable for analysis on a multiplex plate reader were made by Invitrogen for CXCL9, CXCL10, MCP-1, IL-1RA, RANTES, IL-13 and IL-17. 20 ul of freshly thawed BALF supernatant from each sample was incubated with capture beads. Following incubation the protocol for luminex multiplex analysis was followed according to the manufacturer (see cat no LHC6001). All samples and standards were run in duplicate. Chemokine concentrations were obtained using a Luminex 200 instrument (Millipore, Billerica, Mass.).

### D. CXCR3 Analysis

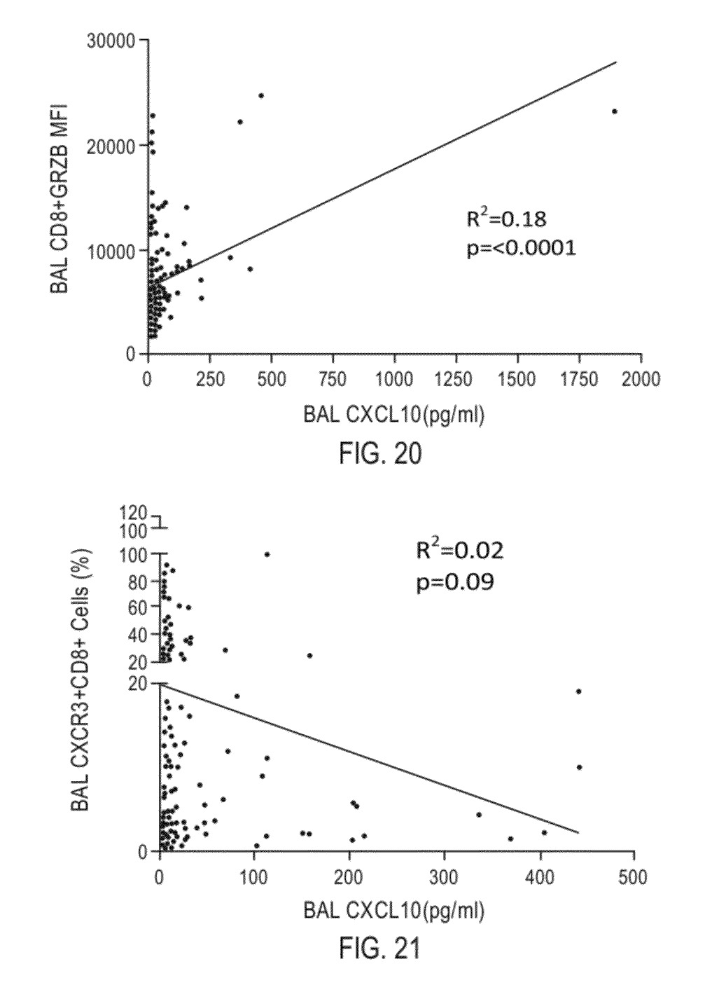

Of the original BAL samples 137/143 had data which included both CD8 GranzymeB, CD8 CXCR3 and histology. We were interested to see if a correlation existed between T cells which had an effector phenotype (CD8 GranzymeB and T cells which expressed the chemokine receptor for CXCL9, CXCL10, and CXCL11, i.e., CXCR3). The expectation was that high levels of CD8 GranzymeB, which correlates with rejection score, would be seen in cases in which there was a high frequency of CD8 CXCR3 positive cells. This assumption was based on the fact that in assessing 146 BAL samples taken from the Emory Lung Transplant Program we had observed a strong correlation between BAL CXCL10 level and CD8 GranzymeB hi frequency (p<0001, r=0.45). In order to assess the relationship between GranzymeB and CXCR3 the non-parametric correlation coefficient was calculated between these two values using data obtained from all recipients who underwent lung transplant between 2007 and 2009. A total of 319 samples were analyzed and a significant negative correlation between BAL CD8 GranzymeB hi and CXCR3 frequencies (p=0.0015, r=−0.18) was observed. FIG. 20 represents the relationship between BAL CXCL10 level and Mean Fluorescence Intensity of BAL CD8+Granzyme B Mean. There is a significant positive correlation between these two markers of lung injury (p<0.05 by Spearman rank correlation). FIG. 21 represents the relationship between BAL CXCL10 and BAL CXCR3+CD8+ cells demonstrating a negative correlation between these makers of lung injury.

### E. BAL Supernatant Assessment

Standard curves of CXCL9, CXCL10, RANTES, IL1-RA, MCP1, IL13, and IL17 were prepared by serially diluting the undiluted standards in Buffer A (PlexMark 3 Kit, Invitrogen. 1× antibody bead stock for each analyte was prepared by diluting the provided 10× antibody bead stock in Working Wash Solution to a final volume of 25 uL. A 96-well filter plate (Invitrogen) was pre-wet with 200 uL of Working Wash Solution and incubated for 30 seconds, followed by aspiration with a vacuum manifold. 25 ul of the diluted antibody bead solution was added to each well. 200 uL of Working Wash Solution was added to the wells and the beads were allowed to soak for 30 seconds. The plate was aspirated and 50 uL of each pre-mixed standard analyte was added to the designated wells. 50 uL of BAL supernatant was added to the sample wells. The plate was incubated at room temperature for 2 hours on an orbital shaker. The plate was then washed two times with Working Wash Solution, and 100 uL of pre-diluted 1× biotinylated detector antibody was added to each well. The plate was incubated for 1 hour at room temperature on an orbital shaker. The plate was washed 2 times with Wash Solution and 100 uL of Streptavidin-RPE (PlexMark 3 Kit) was added to each well, followed by incubation for 30 minutes on an orbital shaker. After 3 more wash steps 100 uL of Wash Solution was added to each well to resuspend the beads. The fluorescence of each analyte in each well was calculated using a Bio-Plex 200 Luminex plate reader (BioRad). Concentrations of each analyte per sample well were calculated by comparison to the standard curve using Bio-Plex Manager 5.0 software (BioRad).

### F. Statistical Analysis

The relevant clinical outcomes of graft survival, freedom from BOS, acute rejection and pre and post-transplant FEV1 improvement were determined a-priori. The exposure for each patient for each chemokines studied was calculated by measuring the concentration of each analyte over the course of a year. We plotted time post-transplant versus chemokines concentration for each patient and calculated the area under this curve (AUC), a unit-less value, for each chemokines assessed by integration. We assessed survival by Kaplan Meier analysis with statistical significance performed using the log-rank test. For assessing the relationship between chemokines AUC and FEV1 measurement we performed non-parametric Spearman correlation. For comparison of rejection to non-rejection biopsies we performed non-parametric paired t-tests. All statistical calculations were performed using Graph Pad Prism 5, La Jolla Calif. A p value of 0.05 or less was considered statistically significant.

### G. Determination of Specificity and Sensitivity

The initial analysis compared rejection and non-rejection samples by non-paired analysis and allowed for calculation of specificity and sensitivity performance characteristic. Subsequently the performance of combinations of the analytes was analyzed. The statistical package of Graph Pad Prism (Graph Pad Software, Inc., La Jolla, Calif.) was used to calculate the performance characteristics for each individual analyte. Using step-wise cutoffs for positive values we calculated receiver-operator curves for each individual analyte where specificity was plotted against 1-sensitivity. The area under the receiver operator curve (AU-ROC or AUC) depicts the test with the best trade off of sensitivity and specificity (Table 2). Additional analyses were calculated to compare combinations of CXCL9, CXCL10, RANTES, and IL1R analytes for specificity and sensitivity. The combination that had the highest specificity and sensitivity was CXCL9+CXCL10 where the cut-off values for each chemokine were CXCL9 (detected) and CXCL10 15 pg/mL.

Table 3 illustrates the BAL scoring array montages:

TABLE 3

| Montage 1 | Montage 2 | Montage 3 | Montage 4 | Montage 5 |
|---|---|---|---|---|
| CXCL10 | CXCL10 (0-3 pts) | CXCL10 (0-3 pts) | CXCL10 (0-3 pts) | CXCL10 (0-3 pts) |
| (0-3 pts) | | | | |
| RANTES | RANTES (0-1 pt) | RANTES (0-1 pt) | RANTES (0-1 pt) | RANTES (0-1 pt) |
| (0-1 pt) | | | | |
| IL1-RA (0-1 pt) | IL1-RA (0-1 pt) | IL1-RA (0-1 pt) | IL1-RA (0-1 pt) | IL1-RA (0-1 pt) |
| CXCL9 (0-1 pt) | CXCL9 (0-1 pt) | CXCL9 (0-1 pt) | CXCL9 (0-1 pt) | CXCL9 (0-1 pt) |
| | % of BAL CD8 | MFI of BAL CD8 | % of BAL CD8 | % of BAL CD8 |
| | Granzyme Bhi | For GranzymeB | Granzyme Bhi | Granzyme Bhi |
| | (0-3 pt) | (0-3 pt) | (0-3 pt) | (0-3 pt) |
| | | | % of BAL CD8 | BAL eosinophils |
| | | | HLADRhiCD38hi | or basophils |
| | | | (effector CD8s) | (0 or 2 pts) |
| 6 possible pts | 9 possible pts | 9 possible pts | 10 possible pts | 11 possible pts |

A comparison of the 5 scoring montages to detect acute rejection in 20 lung transplant recipient cohorts are shown in FIGS. 3A-3E. All rejection episodes in the first post-transplant year were included in this analysis. Total point score is on the y-axis and lung histology scoring is on the x-axis.

Performance characteristics for each of the five scoring montages to detect acute rejection by receiver operator characteristics adapted from the data FIGS. 3A-3E. FIGS. 3F-3J show % sensitivity is on the y-axis and % specificity is on the x-axis, 0% to 100% specificity going from left to right. The AUC of the ROC are 0.78, 0.79, 0.76, 0.78 and 0.83 respectively. Montage 5 gives the best trade off between sensitivity and specificity.

The summary performance characteristics for each scoring montage to detect acute rejection are provided in Table 4, below. The area under the curve (AUC) represents the AUC for the receiver operator curves from FIGS. 3F-3J.

TABLE 4

| Montage | AUC | p value |
|---|---|---|
| 1 | 0.78 | <0.0001 |
| 2 | 0.79 | <0.0001 |
| 3 | 0.76 | <0.0001 |
| 4 | 0.78 | <0.0001 |
| 5 | 0.83 | <0.0001 |

The montages for the scoring arrays were compared in order to correlate with the slope of the FEV1 curve between 9 and 18 months post-transplant. Montage 1 includes only chemokine scores, Montage 2 incorporates chemokine score and CD8 granzymeBhi %, Montage 3 incorporates chemokine score and CD8 granzymeB MFI, Montage 4 incorporates chemokine score, CD8 granzymeB %, and CD8 effector cells, and Montage 5 incorporates chemokine score, CD8 granzyme B, and the presence of eosinophilia in the BAL. FIGS. 3K-3O graphically represent the score for each montage (y axis) vs. the FEV1 slope (x-axis) and the curves represent each of the five montages:

### CXCL9 and CXCL10 Results:

Increased CXCL9 and CXCL10 Exposure is Associated with Increased Graft Loss and Mortality

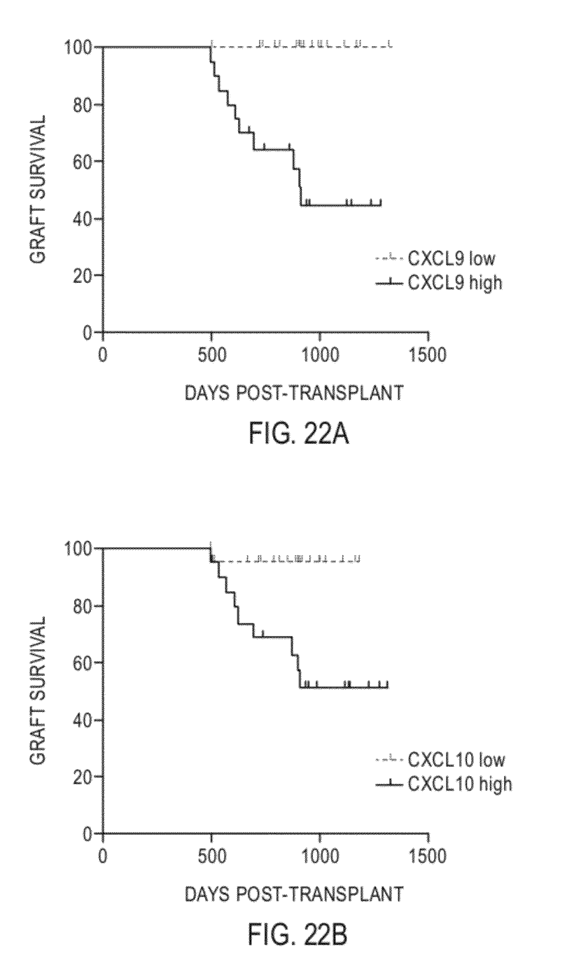

The cumulative BALF exposure was calculated for each analyte in all 40 patients of the cohort by integrating the concentration of each analyte over the time of collection. This generated a chemokine area under the curve (AUC) for the first year post-transplant for each analyte on each patient. The AUC represents a better estimate of cumulative exposure compared with assessing the mean value for each chemokines because the interval between surveillance bronchoscopies is not constant. Because the AUCs for each analyte were not normally distributed among patients the median AUC for each analyte was calculated. The calculation defined a low and high group for each analyte based on whether the AUC value was above or below the median value. For each analyte assessed there were 20 patients in the high group and 20 patients in the low group. Next, the overall graft survival for each analyte was assessed by Kaplan-Meier analysis. A graft was considered failed when the patient died, or went onto retransplantation. Of the 40 patients studied, there were 10 graft failures total (8 deaths and 2 retransplantations). As shown in FIG. 22, Patients with 1-year CXCL9 AUCs above the median for the group were responsible for all of the graft failures. Similarly, patients with 1-year CXCL10 AUCs above the median accounted for 9 of the 10 graft failures. Collectively the hazard ratio (HR) for a high CXCL9 AUC was 9.37 (range 2.69-32.7) and for a high CXCL10 AUC was 5.52 (range 1.59-19.1), p=0.0007 for CXCL9 and p=0.007 for CXCL10. Event free survival indicates freedom from death or listing for retransplantation. P=0.0004 by Log Rank Test. N=20 patients per group. As shown in Table 5, there was a trend toward better survival in patients with low MCP-1 AUC, (HR 2.82, range 0.81-9.8) p=0.0960, but this did not reach statistical significance. There did not appear to be any survival trend for cumulative exposure to IL-17, Rantes, or IL1-RA.

TABLE 5

| | % Graft | % Graft | | |
|---|---|---|---|---|
| | Survival | Survival | | |
| | AUC > | AUC < | HR for AUC > Mean | |
| Chemokine | Median | Mean | (confidence interval | P value |
| CXCL9 | 50% | 100% | 9.37 (2.69-32.7) | .0007 |
| CXCL10 | 55% | 95% | 5.52 (1.59-19.1) | .007 |
| MCP-1 | 65% | 85% | 2.82 (.81-9.8) | .096 |
| Rantes | 65% | 85% | 2.56 (.74-8.94) | .14 |
| IL1-RA | 70% | 80% | 1.46 (.42-5.06) | .56 |
| IL 17 | 80% | 70% | .53 (.15-1.86) | .27 |
| IL 13 | undetected | undetected | NA | NA |

Comparison of graft survival in patients with high or low cumulative exposures to 7 BALF chemokines in the first year post-transplant. BALF IL13 levels were not detectable in our patient samples; survival could not be defined for ID 3 exposure.
Increased CXCL9 and CXCL10 Exposure is Associated with Increased Risk of Early Chronic Rejection

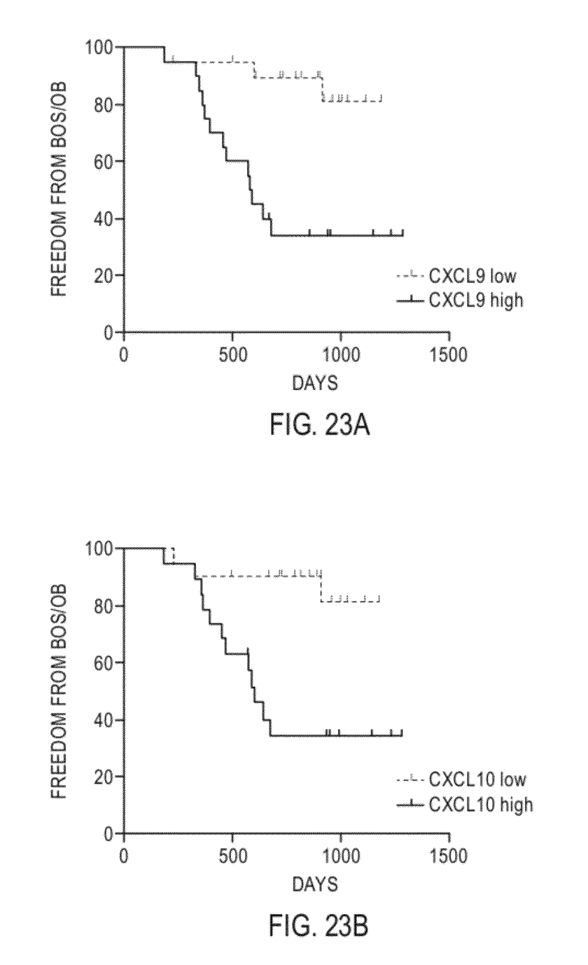

Bronchiolitis Obliterans Syndrome is a surrogate clinical endpoint that approximates histologic BO even when BO has not been detected by biopsy. Similar to the assessment of graft survival, time to first demonstration of BOS or histologic BO (whichever came first) was assessed in the 40 patient cohort. Using this endpoint it was found that 16 of the 40 total patients to have either met the criteria for BOS (a sustained 20% reduction of FEV1 not explained by other causes) or actual histologic confirmation of BO by lung biopsy. As shown in FIG. 23, elevated CXCL9 and CXCL10 AUC over the first year post-transplant was predictive of an increased risk of BO/BOS; HR for CXCL9 5.38 (range 1.97-14.7, p=0.001) and HR for CXCL10 5.21 (range 1.84-14.7, p=0.0018). There was a statistically significant weak effect of MCP-1 as well; HR 2.76 (range 1.02-7.44, p=0.045). Patients were divided into high and low levels if they were above or below the median. Event free survival indicates freedom from diagnosis of BOS or histologic OB. P=0.001 for CXCL9 (FIG. 23A) and 0.002 for CXCL10 (FIG. 23B) by Log Rank Test. N=20 patients per group. As in the graft survival analysis no significant correlation was observed between IL-17, RANTES or IL1RA and freedom from BO/BOS.

Increased CXCL9 and CXCL10 Exposure Correlates with Peak Lung Function Obtained Early After Lung Transplant.

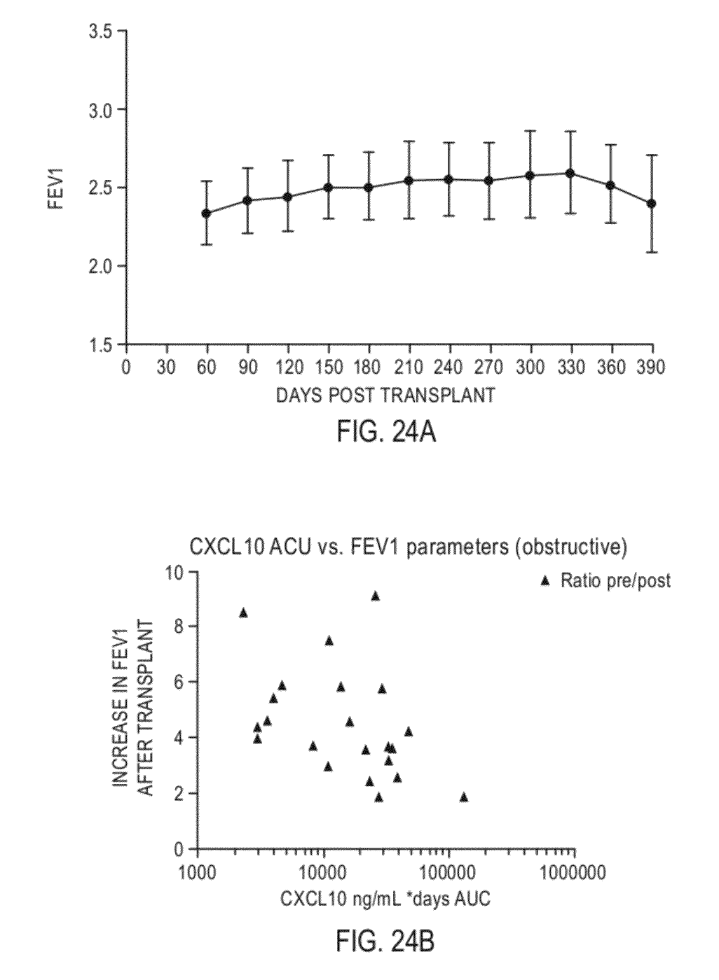

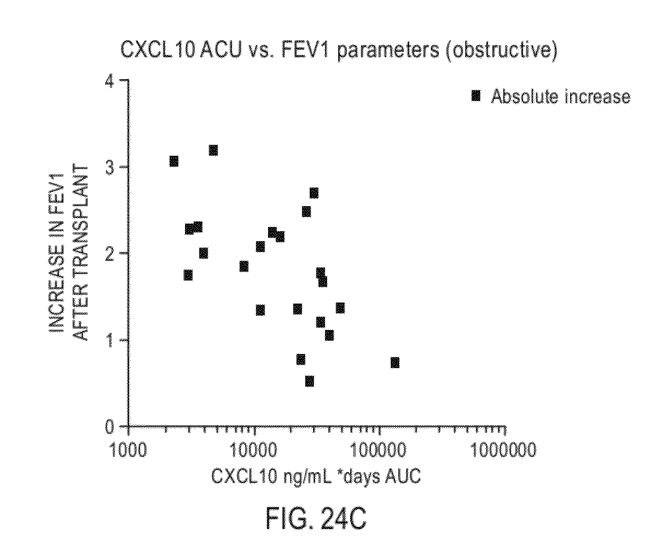

One of the determinants to longevity and quality of life after transplant is the degree of improvement over pre-transplant lung function that is conveyed by transplantation. Undoubtedly there are multiple factors that affect the degree of improvement following transplant which include the type of disease for which the recipient is transplanted, the nature of the transplant (bilateral vs. single), the age and size of the transplant recipient. The extent to which cumulative 1 year exposure to the analytes measured in the multiplex ELISA correlated with peak lung function was assessed. First, the defined peak lung function in the cohort was determined by analyzing the spirometry of the 40 patient cohort as a group. It was found that the highest mean FEV1 was achieved between 270 and 330 days for when the entire group was analyzed (FIG. 24A). Based on this the mean peak FEV1 was calculated for each patient using the mean FEV1 from 270-330 days post-transplant. Peak lung function was achieved at 300 days. The last pre-transplant FEV1 for each patient was used as the pre-transplant baseline. For each patient both the absolute FEV1 increase from pre-transplant to peak post-transplant was calculated as well as the ratio of the peak post-transplant to pre-transplant FEV1. The correlation between each BALF analyte 1-year AUC to the change in lung function pre- and post-transplant was calculated. Because patients with obstructive lung diseases (e.g. emphysema and cystic fibrosis) demonstrated considerably more absolute and relative increases in FEV1 compared to patients with restrictive lung disease (e.g. pulmonary fibrosis, silicosis) patients in each physiologic category (obstructive vs. restrictive) were analyzed separately. As shown in FIGS. 24 B and 24 C, and Table 6 there was strong negative correlation between CXCL9 and CXCL10 AUC and FEV1 changes post-transplant for the 22 patients characterized as having obstructive lung disease, the relationship between CXCL10 AUC and the ratio of the after to before lung function (r=0.5, p=0.018, FIG. 24B), and CXCL10 AUC and the absolute FEV1 improvement post lung transplant (r=−0.57, p-0.006, FIG. 24C). In other words, patients with elevated cumulative levels of CXCL9 or CXCL10 had less pronounced improvements in their FEV1 peak lung function after transplant. As shown in Table 6, the 18 patients with restrictive disease did not demonstrate a statistically significant correlation between CXCL9 AUC and FEV1 parameters, but a significant correlation did exist between CXCL10 AUC and the absolute increase in FEV1 seen in patients with restrictive lung disease.

TABLE 6

| | Correlation | Correlation | | Correlation to |
|---|---|---|---|---|
| | to FEV1 | to FEV1 | | FEV1 |
| | pre/post ratio | pre/post | Correlation | pre/post |
| | obstructive | absolute | to FEV1 | absolute |
| | disease | difference | pre/post ratio | difference |
| | (n = 22) | obstructive | restrictive | restrictive |
| | (r value, | disease | disease | disease |
| Chemokine | p value) | (n = 22) | (n = 18) | (n = 18) |
| CXCL9 | −.61, .0026 | −.61, .0028 | −.18, .48 | −.24, .33 |
| CXCL10 | −.50, .018 | −.57, .006 | −.41, .10 | −.54. 02 |
| MCP-1 | −.44, .04 | −.47, .03 | −.17, .50 | −.34, .14 |
| Rantes | −.04, .84 | −.15, .5 | .07, .78 | .05, .83 |
| IL1-RA | −.15, .5 | −.11, .62 | .11, .66 | .13, .61 |
| IL 17 | .16, .48 | .2, .38 | 1.28, .26 | .23, .33 |
| IL 13 | undetected | undetected | undetected | undetected |

Correlation between cumulative chemokine exposure and improvements in lung function after lung transplant. Shown are the relationships between each chemokine AUC to the ratios of pre-transplant to peak post-transplant FEV1 values or to the absolute increase in FEV1 post-transplant. The Spearman correlation coefficient is shown with the p value for each analysis.

### CXCL9 and CXCL10 are Increased in the Setting of Acute Rejection

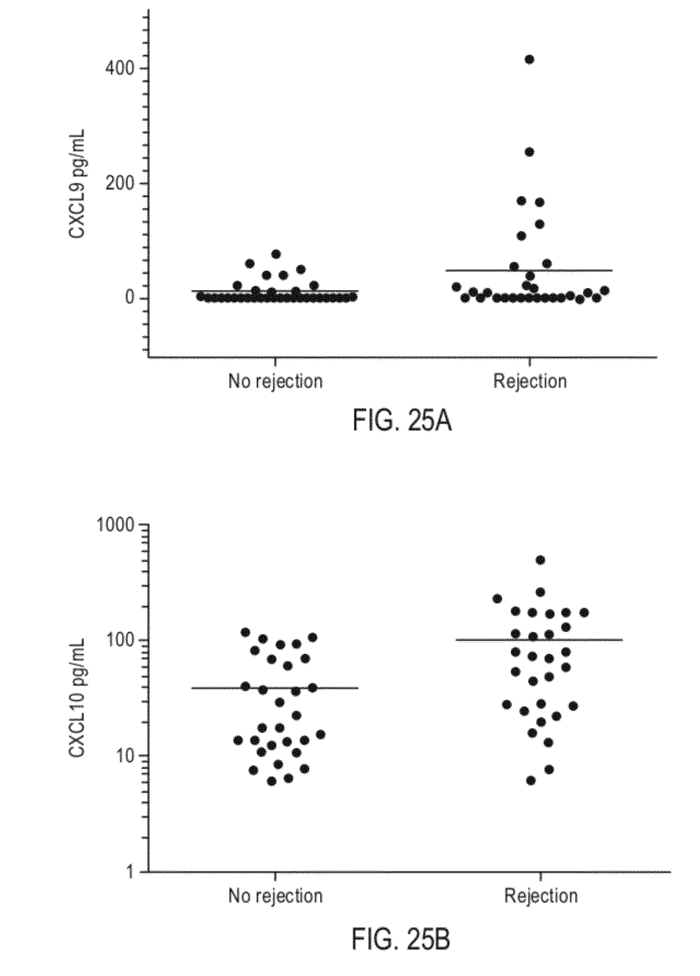

Since the severity of acute rejection is one predictor of the risk of developing BO, how CXCL9 and CXCL10 BALF levels tracked in the setting of acute rejection compared to quiescence were also assessed. Because of significant between patient variability among levels of the chemokines tested comparison of the levels of chemokines within the same patient in the setting of acute rejection vs. quiescence was assessed. In order to mitigate against the potentially confounding effect of a patient having multiple episodes of acute rejection the mean level of each chemokine in rejection and in quiescence was calculate. Rejection was defined as any evidence of venulitis or bronchiolitis (A score or B score) by ISHLT criteria as determined by the pulmonary pathologist. Not surprisingly there was significant variability among the various chemokines among patients in both the setting of rejection and quiescence. Nevertheless, as shown in FIG. 25, CXCL9 and CXCL10 levels were significantly elevated in the setting of ACR compared to quiescent biopsies. When compared to quiescence in a paired analysis with a mean the mean CXCL9 levels were 12 pg/ml in quiescence vs. 50 pg/ml in rejection and the mean CXCL10 levels were 39 pg/ml in quiescence vs. 100 pg/ml in rejection (p=0.0426 and 0.0002, respectively).

### CXCL9 and CXCL10 Expression is Localized to the Bronchiolar Epithelium

Given that CXCL9 and CXCL10 were elevated in the BALF of many samples and correlated with both acute rejection and subsequent risk of chronic rejection, the source(s) of these chemokines was investigated. Because the alveolar macrophage is the predominant cell type recovered during bronchoalveolar lavage and is capable of secreting a large variety of proinflammatory chemokines, it was predicted that staining would be localized to the macrophages present in the intra-alveolar spaces. Immunohistochemistry was performed on transbronchial lung biopsy specimens from 10 patients with the highest BALF levels of CXCL10 and CXCL9 obtained during routine surveillance bronchoscopies. Representative panels are shown in FIG. 26. FIG. 26A shows positive staining for CXCL10 of alveolar macrophages. FIG. 26B shows positive staining of bronchiolar epithelium compared to isotype control antibody. FIG. 26C shows CXCL9 staining of both alveolar macrophages (arrows) and the alveolar epithelium (arrowhead). Consistent with the prediction, alveolar macrophages were found to stain readily for both CXCL9 and CXCL10. Additionally it was noted in biopsy specimens that contained well delineated bronchiolar epithelium significant staining for CXCL9 and CXCL10 and not for control isotype antibody. Further, in some specimens there was additional moderate staining found within the alveolar epithelium. No significant staining of the bronchiolar submucosa or the pulmonary vasculature was observed. Collectively these findings indicate that the elevated BALF CXCL9 and CXCL10 could be derived from both a hematopoietic source (macrophages) and a non hematopoietic source—the small airway epithelium itself.

Those in the art understand that the detection techniques employed are generally not limiting. Rather, a wide variety of detection means are within the scope of the disclosed methods and kits, provided that they allow the presence or absence of an analyte, biomarker or biomolecule to be determined.

While the principles of these teachings have been described in connection with specific embodiments, it should be understood clearly that these descriptions are made only by way of example and are not intended to limit the scope of the teachings. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

All publications and patents mentioned in the above specification are herein incorporated by reference. Various modifications and variations of the described methods and systems of the present teachings will be apparent to those skilled in the art without departing from the scope and spirit of the present teachings. Although the present teachings have been described in connection with specific embodiments, it should be understood that the present teachings as claimed should not be unduly limited to such specific embodiments. Indeed, various modifications of the described modes for carrying out the present teachings that are obvious to those skilled in the relevant fields are intended to be within the scope of the following claims.

What is claimed is:

1. A method of determining chronic and acute cellular rejection in a subject comprising:
(a) contacting a brochoalveolar lavage (BAL) sample with reagents for detection of at least one analyte selected from the group consisting of IL1RA, CXCL10, CXCL11, MCP-1, CXCL9, RANTES, IL-13, IL-17, IL-22, fractalkine, and eotaxin; and
(b) detecting presence or absence of said at least one analyte from the group consisting of IL1-RA, CXCL10, MCP-1, CXCL9, RANTES, IL-13, IL-17, IL-22, fractalkine, and eotaxin in said BAL sample using said reagents,
wherein rejection comprises detecting at least one analyte at least 15 pg/mL.

2. The method of claim 1, further comprising the step of detecting at least one marker selected from the group consisting of CD8 Granzyme B frequency, CD8 GranzymeB mean fluorescence intensity (MFI), CD8 HLA-DR frequency, and CD38 frequency, or at least one cell type selected from eosinophils and basophils.

3. The method of claim 2, wherein CD8 Granzyme B frequency and CD8 HLA-DR, and CD38 frequencies are detected or CD8 Granzyme B frequency and eosinophils or basophils are detected.

4. The method of claim 1, wherein a plurality of BAL samples are analyzed in a longitudinal analysis.

5. The method of claim 4, wherein detecting in at least one sample at least 15 pg/ml CXCL10 and/or detecting CXCL9 indicates a treatment course of action for chronic and acute cellular rejection.

6. The method of claim 5, wherein said amount detected of said at least one analyte from the group consisting of IL1-RA, CXCL10 (IP10), MCP-1, (MIG), RANTES, IL-13, IL-17, IL-22, fractalkine, and eotaxin in said BAL sample is at least 15 pg/ml.

7. The method of claim 6, wherein said detecting further comprises detecting the presence or absence of at least CXCL9 and CXCL10.

8. The method of claim 7, further comprising detecting the presence or absence of at least one of IL1-RA and RANTES.

9. The method of claim 1, wherein said reagents are affixed to a solid support.

10. The method of claim 9, wherein said solid support is selected from the group consisting of a bead, nitrocellulose, a glass slide, a chip, a test strip, and a membrane.

11. The method of claim 10, wherein said solid support is a bead.

12. The method of claim 1, wherein said reagents comprise reagents for performing an immunoassay.

13. The method of claim 12, wherein said immunoassay is selected from the group consisting of an ELISA, radioimmunoassay, automated immunoassay, bead assay, and immunoprecipitation assay.

14. The method of claim 1, wherein said reagents comprise reagents for performing a fluorescently activated cell sorting assay.

15. A method for distinguishing nonrejection from chronic or acute cellular rejection in a subject comprising:
(a) contacting a bronchoalveolar lavage (BAL) sample from the subject with reagents for detection of at least one analyte selected from the group consisting of IL1RA, CXCL10, MCP-1, CXCL9, RANTES, IL-13, IL-17, IL-22, fractalkine, and eotaxin, IL-22, fractalkine, eotaxin; and
(b) detecting presence or absence of said at least one analyte from the group consisting of IL1-RA, CXCL10, MCP-1, CXCL9, RANTES, IL-13, IL-17, IL-22, fractalkine, and eotaxin in said BAL sample using said reagents.

16. The method of claim 15, wherein detecting said at least one analyte in said BAL sample distinguishes nonrejection from chronic or acute cellular rejection.

17. The method of claim 15, further comprising detecting at least one marker selected from the group consisting of CD8 Granzyme B frequency, CD8 GranzymeB MFI, CD8 HLA-DR frequency, CD38 frequency, or detecting at least one cell type selected from eosiniophils and basophils.

18. The method of claim 17, wherein detecting CD8 Granzyme B frequency and CD8 HLA-DR and CD38 frequencies or CD8 GranzymeB frequency and eosinophils or basophils in said BAL sample distinguishes nonrejection from chronic or acute cellular rejection.

19. The method of claim 15, wherein a plurality of BAL samples are analyzed in a longitudinal analysis.

20. The method of claim 15, wherein said detecting the presence or absence of said at least one analyte from the group consisting of IL1-RA, CXCL10 (IP10), MCP-1, CXCL9 (MIG), RANTES, IL-13, IL-17, IL-22, fractalkine, and eotaxin in said BAL sample using said reagents in distinguishing nonrejection verses chronic or acute cellular rejection comprises detecting at least 15 pg/ml of at least one analyte from the group consisting of IL1-RA, CXCL10, MCP-1, RANTES, IL-13, IL-17, IL-22, fractalkine, and eotaxin and CXCL9.

21. The method of claim 20, wherein said detecting comprises detecting the presence or absence of at least CXCL9 and CXCL10.

22. The method of claim 21, further comprising detecting the presence or absence of at least one of IL1-RA and RANTES.

23. The method of claim 15, wherein said reagents are affixed to a solid support.

24. The method of claim 24, wherein said solid support is selected from the group consisting of a bead, nitrocellulose, a glass slide, a chip, a test strip, and a membrane.

25. The method of claim 25, wherein said solid support is a bead.

26. The method of claim 15, wherein said reagents comprise reagents for performing an immunoassay.

27. The method of claim 27, wherein said immunoassay is selected from the group consisting of an ELISA, radioimmunoassay, automated immunoassay, bead assay, and immunoprecipitation assay.

28. The method of claim 15, wherein said reagents comprise reagents for performing a fluorescently activated cell sorting assay.

29. A method of detecting disorders of the lung, comprising:
(a) contacting a bronchoalveolar lavage (BAL) sample with reagents for detection of at least one analyte selected from the group consisting of IL1RA, CXCL10, MCP-1, CXCL9, RANTES, IL-13, IL-17, IL-22, fractalkine, and eotaxin, IL-22, fractalkine, eotaxin; and
(b) detecting presence or absence of said at least one analyte from the group consisting of IL1-RA, CXCL10, MCP-1, CXCL9, RANTES, IL-13, IL-17, IL-22, fractalkine, and eotaxin in said BAL sample using said reagents, wherein detecting at least 15 pg/ml of said at least one analyte indicates disorder of the lung.

30. The method of claim 29, wherein a plurality of BAL samples are evaluated in a longitudinal analysis.

31. The method of claim 29, wherein said amount of said analyte in said BAL sample is at least 15 pg/ml.

32. The method of claim 29, wherein said amount of said analyte in said BAL sample is at least 30 pg/ml.

33. The method of claim 29, wherein said amount of said compound in said BAL sample is at least 50 pg/ml.

34. The method of claim 29, wherein said reagents are affixed to a solid support.

35. The method of claim 34, wherein said solid support is selected from the group consisting of a bead, nitrocellulose, a glass slide, a chip, a test strip, and a membrane.

36. The method of claim 35, wherein said solid support is a bead.

37. The method of claim 29, wherein said reagents comprise reagents for performing an immunoassay.

38. The method of claim 37, wherein said immunoassay is selected from the group consisting of an ELISA, radioimmunoassay, automated immunoassay, bead assay, and immunoprecipitation assay.

39. The method of claim 29, wherein said reagents comprise reagents for performing a fluorescently activated cell sorting assay.

40. The method of 38, wherein said immunoassay is a bead assay.

41. The method of claim 29, further comprising the step of determining the presence or absence of a concurrent infection in said subject.

42. The method of claim 29, further comprising detecting one or more of CXCL9, IL-RA, and RANTES.

43. The method of claim 29, further comprising detecting one or more of IL-13, IL-17, MCP-1, CD8 Granzyme B frequency, CD8 GranzymeB MFI, CD8 HLA-DR frequency, CD38 frequency, eosiniophils and basophils.

44. The method of claim 43, wherein said CD8 Granzyme B frequency, CD8 GranzymeB MFI, CD8 HLA-DR frequency and CD38 frequency, are detected by measuring CD8 Granzyme B, CD8 GranzymeB MFI, and CD8 HLA-DR, and CD38 cells.

45. A method of determining future vitality of a transplanted lung comprising:
contacting a BAL sample from a lung transplant recipient with a reagent for the detection of CXCL10; and
detecting in the BAL sample CXCL10 using said reagent,
wherein detection of at least more than 15 pg/ml of CXCL10 in said BAL sample indicates the recipient is at risk for bronchiolitis obliterans syndrome (BOS) and/or concurrent acute cellular rejection (ACR), and
wherein development of BOS or ACR is adverse to future vitality of the transplanted lung.

46. The method of claim 45, wherein said reagent comprises at least one reagent for performing an immunoassay.

47. The method of claim 46, wherein said immunoassay is selected from the group consisting of an ELISA, radio-immunoassay, automated immunoassay, bead assay, and immunoprecipitation assay.

48. The method of claim 45, wherein said reagent comprises at least one reagent for performing a bead assay.

49. A method of determining the risk of acute lung transplant rejection in a subject who has undergone a lung transplant, comprising:
a) reacting a BAL sample from said subject with reagents for detection of CXCL10; and
b) determining an amount of CXCL10 in said BAL sample using said reagents;
wherein the amount of CXCL10 in said sample is above a cut-off level of 15 pg/mL, said subject is at increased risk for acute lung transplant rejection.

50. The method of claim 49, wherein said reagents comprise reagents for performing an immunoassay.

51. The method of claim 50, wherein said immunoassay is selected from the group consisting of an ELISA, radioimmunoassay, automated immunoassay, bead assay, and immunoprecipitation assay.

52. The method of claim 51, wherein said ELISA is a quantitative ELISA assay.

53. The method of claim 49, wherein said reagents comprise reagents for performing a fluorescently activated cell sorting assay.

54. The method of claim 53, wherein said fluorescently activated cell sorting assay is a quantitiative fluorescently activated cell sorting assay.

55. A method of determining acute lung transplant rejection in a subject who has undergone a lung transplant, comprising:
a) reacting a BAL sample from said subject with reagents for detection of CXCL10; and
b) determining an amount of CXCL10 in said BAL sample using said reagents;
wherein the amount of CXCL10 in said sample is above a cut-off level of 15 pg/mL, said subject is determined to have acute lung transplant rejection.

56. The method of claim 55, further comprising detecting the presence of CXCL9, IL-1-RA, IL-17, RANTES and MCP1 in said BAL sample.

57. The method of claim 55, wherein said reagents comprise reagents for performing an immunoassay.

58. The method of claim 57, wherein said immunoassay is selected from the group consisting of an ELISA, radioimmunoassay, automated immunoassay, bead assay, and immunoprecipitation assay.

59. The method of claim 58, wherein said ELISA is a quantitative ELISA assay.

60. A method of determining a treatment course of action for a subject who has undergone a lung transplant, comprising:
a) reacting reagents for detection of CXCL10 with a BAL sample from said subject, and
b) detecting an amount of CXCL10 in said BAL sample using said reagents;
wherein the amount of CXCL10 in said sample is above a cut-off level of 15 pg/mL, then said subject is determined to be at increased probability of having acute lung transplant rejection; and
c) determining a treatment course of action for said subject based on said increased probability of acute lung transplant rejection.

61. The method of claim 60, wherein said treatment course of action comprises the administration of augmented anti-rejection therapy to said subject such as the decision to administer pulse doses of intravenous corticosteriod therapy based on results in claim 60.

63. The method of claim 60, wherein said treatment course of action comprises continued monitoring of said subject.

64. The method of claim 60, wherein said reagents comprise reagents for performing an immunoassay.

65. The method of claim 64, wherein said immunoassay is selected from the group consisting of an ELISA, radioimmunoassay, automated immunoassay, bead assay, and immunoprecipitation assay.

66. The method of claim 65, wherein said ELISA is a quantitative ELISA assay.

67. The method of claim 60, further comprising determining the presence or absence of a concurrent infection in said subject.

68. The method of claim 67, wherein said determining comprises determining the body temperature of said subject.

69. The method of claim 67, wherein said determining comprises the detection of a bacterial infection in said subject.

70. The method of claim 67, wherein said determining comprises the detection of a viral infection in said subject.

71. A kit for determination of transplant rejection comprising:
reagents for detection of CXCL10 and at least one analyte selected from the group consisting of IL1RA, MCP-1, CXCL9, RANTES, IL-13, IL-17, IL-22, fractalkine, and eotaxin.

72. The kit of claim 71, further comprising reagents for detection of the frequency of at least one marker from the group consisting of CD8 Granzyme B, CD8 GranzymeB MFI, CD8 HLA-DR, and CD38, and/or for detection of a cell type from the group consisting of eosinophils and basophils.

73. The kit of claim 71, wherein said reagents are affixed to a solid support.

74. The kit of claim 73, wherein said solid support is selected from the group consisting of a bead, nitrocellulose, a glass slide, a chip, a test strip, and a membrane.

75. The kit of claim 74, wherein said solid support is a bead.

76. The kit of claim 71, wherein said reagents comprise reagents for performing an immunoassay.

77. The kit of claim 76, wherein said immunoassay is selected from the group consisting of an ELISA, radioimmunoassay, automated immunoassay, bead assay, and immunoprecipitation assay.

78. The kit of claim 71, wherein said reagents comprise reagents for performing a fluorescently activated cell sorting assay.

79. The kit of claim 77, wherein said reagents comprise reagents for performing an ELISA assay.

80. The kit of claim 72, wherein said markers and cells are detected by flow cytometry.

81. A method of determining chronic and acute cellular rejection in a subject comprising:
a. contacting a BAL sample from a subject with a first reagent for detection of at least one chemokine receptor selected from the group consisting of CXCn, CCRn, CX₃CR1, and XC,
wherein n is an integer from 1 to 10;
b. detecting presence or absence of said at least one receptor from the group consisting of CXCn, CCRn, CX₃CR1, and XC in said biopsy sample using said reagent; and
c. determining chronic or acute cellular rejection in said subject based on the result of said detecting.

82. The method of claim 81, further comprising the step of detecting at least one additional receptor selected from the group consisting of CXCn, CCRn, CX₃CR1, and XC with a second reagent for detection of at least one chemokine receptor selected from the group consisting of CXCn, CCRn, CX₃CR1, and XC.

83. The method of claim 82, wherein presence of at least two receptors selected from CXCn, CCRn, CX₃CR1, and XC are detected.

84. The method of claim 81, wherein the CXCn receptor is selected from CXC1, CXC2, CXC3, CXC4, CXC5, CXC6, and CXC7.

85. The method of claim 81, wherein the CCRn receptor is selected from CCR1, CCR2, CCR3, CCR4, CCR5, CCR6, CCR7, CCR8, CCR9, and CCR10.

86. The method of claim 81, further comprising detecting the presence or absence of a chemokine for said at least one chemokine receptor in said biopsy sample.

87. The method of claim 86, further comprising a third reagent for detection of the presence or absence of said chemokine.

88. The method of claim 87, wherein said chemokine is selected from the group consisting of IL1-RA, CXCL10, MCP-1, CXCL9, RANTES, IL-13, IL-17, IL-22, fractilkine, and eotaxin in said biopsy sample using said third reagent.

89. The method of claim 81, wherein said detecting the presence or absence of said at least one receptor from the group consisting of CXCn, CCRn, CX₃CR1, and XC in said BAL sample using said reagents in determining said detecting chronic or acute cellular rejection comprises detecting the presence or absence of said at least one receptor from the group consisting of CXCn, CCRn, CX₃CR1, and XC in said biopsy sample.

90. The method of claim 89, wherein said receptor detected is a chemokine receptor.

91. The method of claim 90, wherein said amount detected of said at least one chemokine receptor from the group consisting of CXCn, CCRn, CX₃CR1, and XC in said BAL fluid is inversely proportional to the amount of a chemokine present for said chemokine's receptor.

92. The method of claim 89, wherein said detecting comprises detecting the presence or absence of at least the chemokine receptors for CXCL9 and CXCL10.

93. The method of claim 89, further comprising detecting the presence or absence of at least one receptor for at least one of IL1-RA and RANTES.

94. The method of claim 81, wherein said biopsy sample is affixed to a solid support.

95. The method of claim 94, wherein said solid support is selected from the group consisting of a bead, nitrocellulose, a glass slide, a chip, a test strip, and a membrane.

96. The method of claim 95, wherein said solid support is a bead.

97. The method of claim 81, wherein said reagents comprise reagents for performing a histology analysis.

98. The method of claim 97, wherein said histology analysis is selected from the group consisting of light microscopy, electron microscopy, and historadiography.
